(19) United States
(12) Patent Application Publication
(10) Pub. No.: US 2023/0250098 A1
(43) Pub. Date: Aug. 10, 2023

(54) KRATOM OPIOID DERIVATIVES FOR THE TREATMENT OF ALCOHOL USE DISORDER

(71) Applicants: University of Health Sciences & Pharmacy in St. Louis, St. Louis, MO (US); Purdue Research Foundation, West Lafayette, IN (US); The Trustees of Columbia University in the City of New York, New York, NY (US)

(72) Inventors: Susruta MAJUMDAR, St. Louis, MO (US); Richard M. VAN RIJN, West Lafayette, IN (US); Soumen CHAKRABORTY, St. Louis, MO (US); Balazs VARGA, St. Louis, MO (US); Anna M. GUTRIDGE, West Lafayette, IN (US); Dalibor SAMES, New York, NY (US)

(73) Assignees: University of Health Sciences & Pharmacy in St. Louis, St. Louis, MO (US); Purdue Research Foundation, West Lafayette, IN (US); The Trustees of Columbia University in the City of New York, New York, NY (US)

(21) Appl. No.: 17/938,003

(22) Filed: Oct. 4, 2022

Related U.S. Application Data
(60) Provisional application No. 63/251,978, filed on Oct. 4, 2021.

(51) Int. Cl. C07D 471/14; C07D 471/20; A61P 25/32

(52) U.S. Cl. CPC C07D 471/14; C07D 471/20; A61P 25/32

(57) ABSTRACT

In some aspects, the present disclosure provides compounds of the formula:

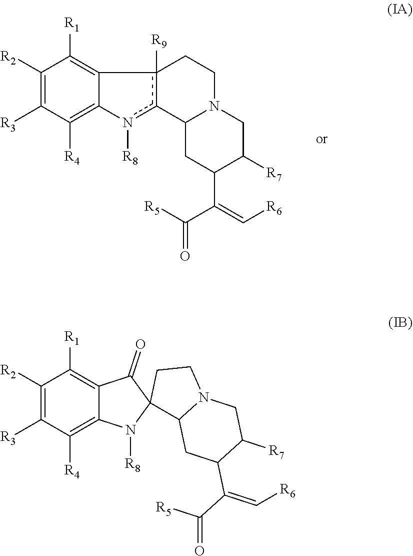

wherein the variables are as defined herein. In some embodiments, these compounds may be used to reduce the consumption of alcohol in a patient. These compounds may be used in treat or prevent alcoholism or an alcohol abuse disorder and show an improved pharmaceutical profile relative to other commonly used compounds.

This application claims the benefit of priority to U.S. Provisional Application No. 63/251,978, filed on Oct. 4, 2021, the entire contents of which are incorporated herein by reference.

The invention was made with government support under Grant Nos. DA045897, DA045884, AA025368, AA026949, and AA026675 awarded by the National Institutes of Health. The government has certain rights in the invention.

### BACKGROUND

### 1. Field

The present disclosure relates generally to the field of pharmaceuticals and active pharmaceutical ingredients. In particular, the compounds described herein may be used to treat an alcohol use disorder.

### 2. Description of Related Art

Mitragyna speciosa, more commonly known as Kratom, is growing increasingly popular in the United States, with nearly 1% of the population age 12 and older using kratom in 2019 (Palamar, 2021). While kratom is most commonly used to self-manage pain or reduce dependence to opioids and opiates, a recent online survey revealed 18% of kratom users indicate reducing or quitting alcohol consumption is a reason they use kratom (Coe et al., 2019). This indication is in line with reports of individuals claiming that kratom was useful for reducing their alcohol intake (Havemann-Reinecke, 2011; Suhaimi et al., 2021; Singh et al., 2014). The systemic injections of kratom extract and kratom alkaloids (7-hydroxymitragynine, paynantheine, speciogynine, mitragynine) have been previously shown to decrease voluntary alcohol drinking in mouse models of moderate and binge alcohol consumption, with the kratom alkaloid 7-hydroxymitragynine being the most efficacious (Gutridge et al., 2020). Kratom alkaloids differ from opium-derived opioids and clinically used synthetic opioids in that upon binding to opioid receptors they activate the Gα_{i/o} protein, without promoting β-arrestin recruitment to the receptor (Kruegel et al., 2016; Váradi et al., 2016; Chakraborty and Majumdar, 2021; Faouzi et al., 2020). 7-hydroxymitragynine and other kratom alkaloids poorly recruit β-arrestin at the μOP and δOP and possess a degree of G-protein bias at this receptor (Gutridge et al., 2020). Moreover, studies in δOP knockout mice revealed that 7-hydroxymitragynine's modulation of alcohol consumption was due to its activity at the SOP (Gutridge et al., 2020). This is in agreement with prior preclinical studies in mice that strongly suggest that β-arrestin recruitment at the delta opioid receptor (SOP) is a liability for enhanced alcohol use and should be avoided (Gutridge et al., 2020; Chiang et al., 2016; Robins et al., 2018).

A possible concern is that 7-hydroxymitragynine and other kratom alkaloids generally have comparable if not higher affinity and potency at the μOP (Takayama et al., 2002; Matsumoto et al., 2004). While this μOP potency may be responsible for the alkaloids' ability to promote antinociception in mice (Matsumoto et al., 2004; Obeng et al., 2020; Wilson et al., 2020; Wilson et al., 2021) and in humans (Vicknasingam et al., 2020), it appears that because of their μOP potency, kratom alkaloids, especially 7-hydroxymitragynine, are shown or predicted to share some of the same negative side effects associated with traditional opioids such as respiratory depression and abuse liability. Accordingly, in rodent preclinical studies, 7-hydroxymitragynine has been shown to have rewarding qualities in models of conditioned place preference and self-administration, which indicates it may have abuse liability (Gutridge et al., 2020; Hemby et al., 2019; Yue et al., 2018). Likewise, withdrawal symptoms following kratom exposure has also been recorded in rodents (Wilson et al., 2021; Matsumoto et al., 2005). Similarly, regular kratom use in humans leads to dependence problems in over 50% of users (Singh et al., 2014), and kratom withdrawal symptoms equally have been widely reported in humans (Singh et al., 2014; Stanciu et al., 2019; Anand & Hosanagar, 2021; Saref et al., 2019). Likely attributed to its potency at the μOP, another side effect of 7-hydroxymitragynine in mice is hyperlocomotion (Gutridge et al., 2020; Becker et al., 2000); this effect mirrors one of kratom's traditional uses as a stimulant (Suwanlert, 1975; Ahmad & Aziz, 2012). Still, relative to traditional opioids such as morphine, the negative side effect profile of kratom and kratom opioids is slightly lessened in regards to reward, respiratory depression, and withdrawal symptoms (Wilson et al., 2020; Wilson et al., 2021; Hemby et al., 2019). This reduction in the side effect profile was first attributed to G-protein biased activity of the kratom alkaloids at the μOP (Kruegel et al., 2016; Váradi et al., 2016), but new research suggests that partial agonism at the μOP likely drives these effects (Gillis et al., 2020; Uprety et al., 2021; Bhowmik et al., 2021). Despite the reduced μOP-mediated side effects relative to traditional opioids, kratom use is not without risk, and this is reflected in controversial efforts to place 7-hydroxymitragynine and mitragynine under Schedule I regulation by the Drug Enforcement Agency (Griffin & Webb, 2018).

An additional side effect of kratom use is seizure activity (Coonan & Tatum, 2021). In rats, abnormal EEG activity has been reported following chronic exposure to mitragynine, the most abundant alkaloid in kratom (Suhaimi et al., 2021). In humans, several individual case reports have highlighted seizure side effects induced by kratom use or withdrawal (Burke et al., 2019; Boyer et al., 2008; Tatum et al., 2018; Valenti et al., 2021; Afzal et al., 2020; Nelson et al., 2010), and retrospective analysis of kratom exposure reports to the National Poison Data System reveals that 6.1% of reports detail seizure side-effects (Eggleston et al., 2019). Currently the mechanism underlying these reported kratom's seizure effects have not been defined.

Therefore, a need to develop new compounds that show favorable opioid receptor activation remains.

### SUMMARY

In some aspects, the present disclosure provides compounds which are kratom alkaloid derivatives which show an improved pharmaceutical profile such as a broadened therapeutic window or decreased alcohol consumption in subjects. In some embodiments, the compounds described herein may have reduced potency at the mu opioid receptor (μOP). Without wishing to be bound by any theory, it is believed that these compounds lead to delta opioid receptor (SOP) dependent and/or G-biased signaling without additional addictive or respiratory related side effects.

In some embodiments, the compounds are further defined as:

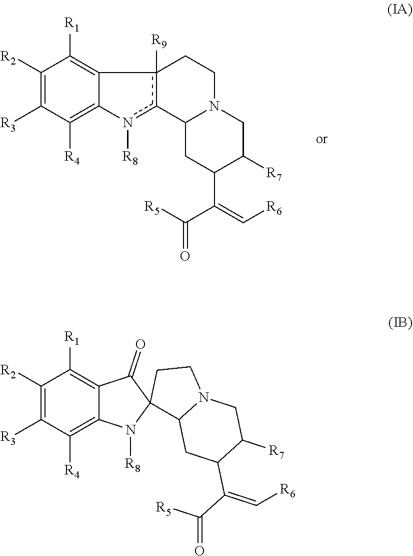

wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₅ is NR′R″ or OR′″ wherein:
   R′ and R″ are each independently hydrogen, alkyl_{(C≤8)}, alkenyl_{(C≤8)}, aryl_{(C≤8)}, aralkyl_{(C≤8)}, or a substituted version of any of those groups; a monovalent amine protecting group, or R′ and R″ are taken together and are a divalent amine protecting group;
   R′″ is hydrogen, alkyl_{(C≤8)}, alkenyl_{(C≤8)}, aryl_{(C≤8)}, aralkyl_{(C≤8)}, or a substituted version of any of those groups; or a hydroxy protecting group,
R₆ is alkoxy_{(C≤12)} or substituted alkoxy_{(C≤12)};
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
provided that the compound is not a compound of the formula:

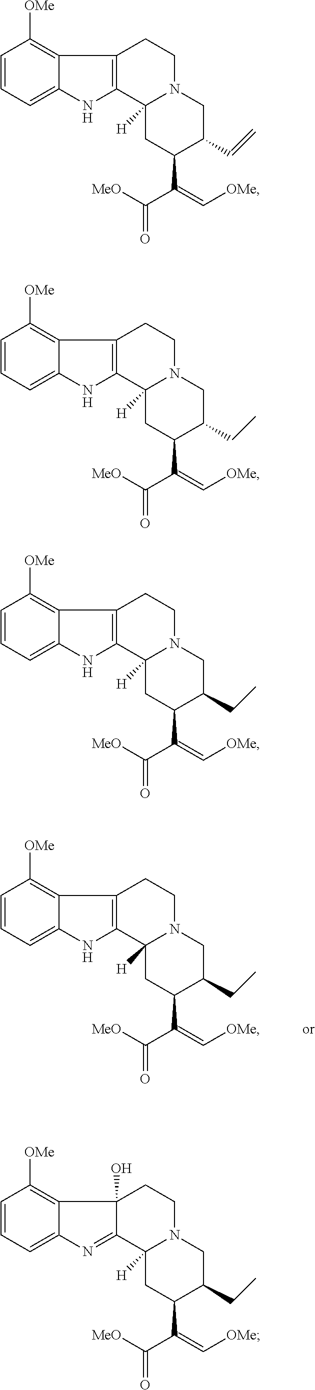 [SMILES: C=C[C@H]1CN2CCc3c([nH]c4cccc(OC)c34)[C@@H]2C[C@@H]1/C(=C\OC)C(=O)OC ; CC[C@H]1CN2CCc3c([nH]c4cccc(OC)c34)[C@@H]2C[C@@H]1/C(=C\OC)C(=O)OC ; CC[C@@H]1CN2CCc3c([nH]c4cccc(OC)c34)[C@@H]2C[C@@H]1/C(=C\OC)C(=O)OC ; CC[C@@H]1CN2CCc3c([nH]c4cccc(OC)c34)[C@H]2C[C@@H]1/C(=C\OC)C(=O)OC ; CC[C@@H]1CN2CC[C@@]3(O)C(=Nc4cccc(OC)c43)[C@@H]2C[C@@H]1/C(=C\OC)C(=O)OC]

or a pharmaceutically acceptable salt thereof.

In some embodiments, the compounds are further defined as:

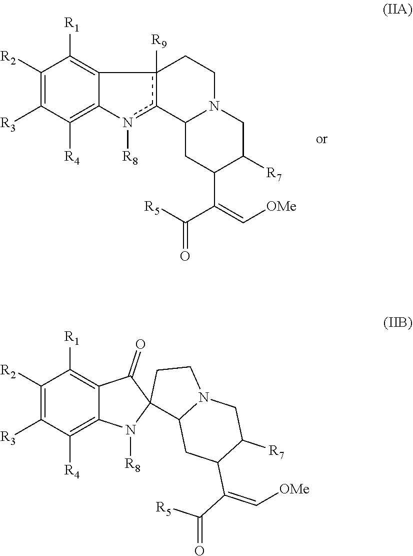

wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₅ is NR′R″ or OR′″ wherein:
   R′ and R″ are each independently hydrogen, alkyl_{(C≤8)}, alkenyl_{(C≤8)}, aryl_{(C≤8)}, aralkyl_{(C≤8)}, or a substituted version of any of those groups; a monovalent amine protecting group, or R′ and R″ are taken together and are a divalent amine protecting group;
   R′″ is hydrogen, alkyl_{(C≤8)}, alkenyl_{(C≤8)}, aryl_{(C≤8)}, aralkyl_{(C≤8)}, or a substituted version of any of those groups; or a hydroxy protecting group,
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
   or a pharmaceutically acceptable salt thereof.

In some embodiments, the compounds are further defined as:

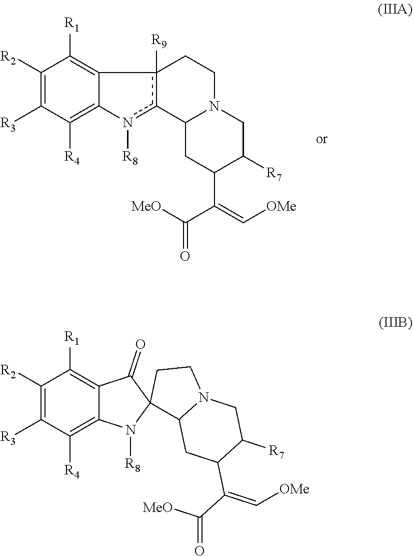

wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
   or a pharmaceutically acceptable salt thereof.

In some embodiments, the compounds are further defined as:

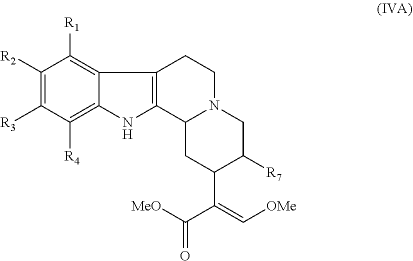

wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
   or a pharmaceutically acceptable salt thereof.

In some embodiments, the compounds are further defined as:

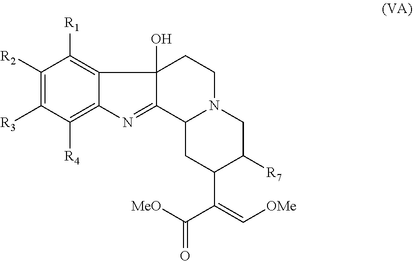

wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
   or a pharmaceutically acceptable salt thereof.

In some embodiments, the compounds are further defined as:

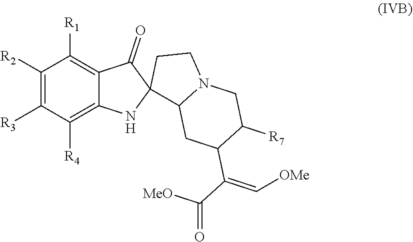

wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
   or a pharmaceutically acceptable salt thereof.

In some embodiments, R₆ is alkoxy_{(C≤12)}. In some embodiments, R₆ is alkoxy_{(C≤6)} such as methoxy. In some embodiments, R₅ is OR′″. In some embodiments, R′″ is alkyl_{(C≤8)} or substituted alkyl_{(C≤8)}. In some embodiments, R′″ is alkyl_{(C≤8)} such as methyl. In other embodiments, R′″ is hydrogen. In other embodiments, R₅ is NR′R″. In some embodiments, R′ is alkyl_{(C≤8)} or substituted alkyl_{(C≤8)}. In some embodiments, R′ is alkyl_{(C≤8)} such as methyl. In other embodiments, R′ is hydrogen. In some embodiments, R″ is alkyl_{(C≤8)} or substituted alkyl_{(C≤8)}. In some embodiments, R″ is alkyl_{(C≤8)} such as methyl. In other embodiments, R″ is hydrogen.

In some embodiments, R₉ is absent. In other embodiments, R₉ is hydroxy. In some embodiments, R₈ is absent. In other embodiments, R₈ is hydrogen. In some embodiments, R₇ is alkyl_{(C≤12)} or substituted alkyl_{(C≤12)}. In some embodiments, R₇ is alkyl_{(C≤12)}. In some embodiments, R₇ is alkyl_{(C≤6)} such as ethyl. In some embodiments, R₇ is alkenyl_{(C≤12)} or substituted alkenyl_{(C≤12)}. In some embodiments, R₇ is alkenyl_{(C≤12)}. In some embodiments, R₇ is alkenyl_{(C≤6)} such as ethylenyl.

In some embodiments, R₁ is alkoxy_{(C≤12)} or substituted alkoxy_{(C≤12)}. In some embodiments, R₁ is alkoxy_{(C≤12)}. In some embodiments, R₁ is alkoxy_{(C≤6)} such as methoxy. In some embodiments, R₁ is hydrogen. In some embodiments, R₂ is alkoxy_{(C≤12)} or substituted alkoxy_{(C≤12)}. In some embodiments, R₂ is alkoxy_{(C≤12)}. In some embodiments, R₂ is alkoxy_{(C≤6)} such as methoxy. In some embodiments, R₂ is hydrogen. In some embodiments, R₃ is alkoxy_{(C≤12)} or substituted alkoxy_{(C≤12)}. In some embodiments, R₃ is alkoxy_{(C≤12)}. In some embodiments, R₃ is alkoxy_{(C≤6)} such as methoxy. In some embodiments, R₃ is hydrogen. In some embodiments, R₄ is alkoxy_{(C≤12)} or substituted alkoxy_{(C≤12)}. In some embodiments, R₄ is alkoxy_{(C≤12)}. In some embodiments, R₄ is alkoxy_{(C≤6)} such as methoxy. In some embodiments, R₄ is hydrogen.

In some embodiments, the compounds are further defined as:

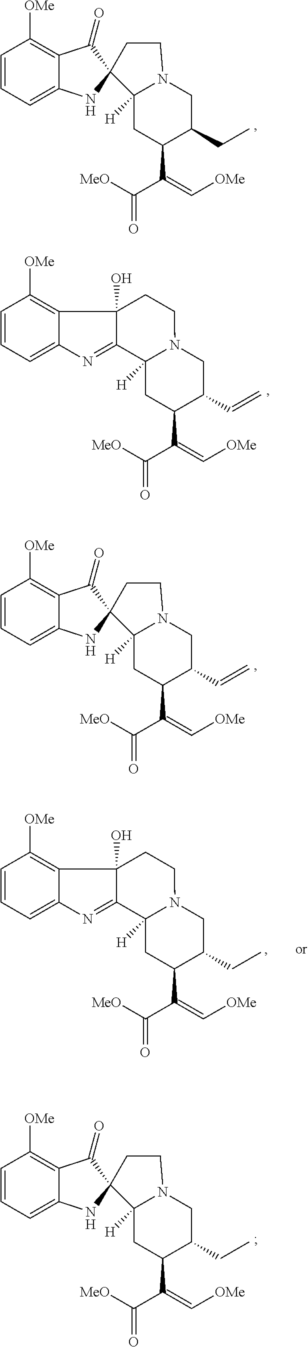 [SMILES: CC[C@@H]1CN2CC[C@]3(Nc4cccc(OC)c4C3=O)[C@@H]2C[C@@H]1/C(=C\OC)C(=O)OC ; C=C[C@H]1CN2CC[C@@]3(O)C(=Nc4cccc(OC)c43)[C@@H]2C[C@@H]1/C(=C\OC)C(=O)OC ; C=C[C@H]1CN2CC[C@]3(Nc4cccc(OC)c4C3=O)[C@@H]2C[C@@H]1/C(=C\OC)C(=O)OC ; CC[C@H]1CN2CC[C@@]3(O)C(=Nc4cccc(OC)c43)[C@@H]2C[C@@H]1/C(=C\OC)C(=O)OC ; CC[C@H]1CN2CC[C@]3(Nc4cccc(OC)c4C3=O)[C@@H]2C[C@@H]1/C(=C\OC)C(=O)OC]

or a pharmaceutically acceptable salt thereof.

In still yet another aspect, the present disclosure provides pharmaceutical compositions comprising:

(A) a compound described herein; and
(B) an excipient,

In yet another aspect, the present disclosure provides pharmaceutical compositions comprising:

(A) a compound of the formula:

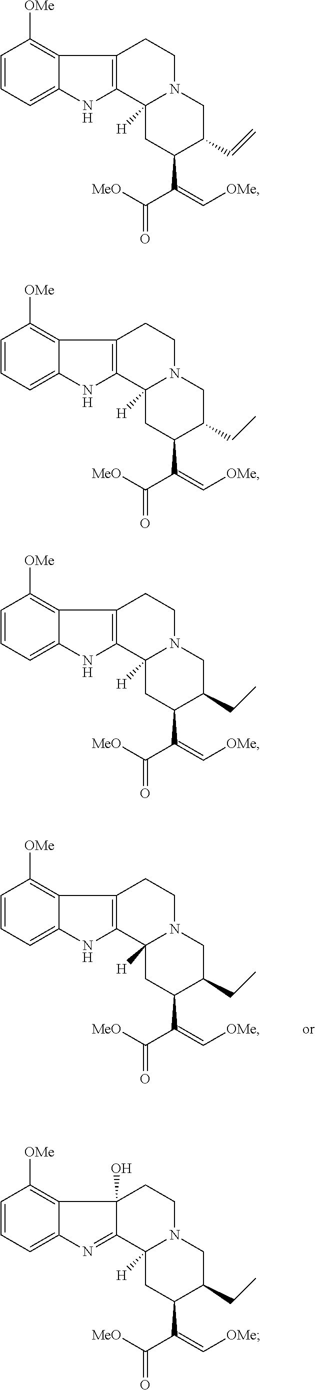 [SMILES: C=C[C@H]1CN2CCc3c([nH]c4cccc(OC)c34)[C@@H]2C[C@@H]1/C(=C\OC)C(=O)OC ; CC[C@H]1CN2CCc3c([nH]c4cccc(OC)c34)[C@@H]2C[C@@H]1/C(=C\OC)C(=O)OC ; CC[C@@H]1CN2CCc3c([nH]c4cccc(OC)c34)[C@@H]2C[C@@H]1/C(=C\OC)C(=O)OC ; CC[C@@H]1CN2CCc3c([nH]c4cccc(OC)c34)[C@H]2C[C@@H]1/C(=C\OC)C(=O)OC ; CC[C@@H]1CN2CC[C@@]3(O)C(=Nc4cccc(OC)c43)[C@@H]2C[C@@H]1/C(=C\OC)C(=O)OC]

(B) an excipient.

In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the pharmaceutical compositions are formulated as a unit dose.

In yet another aspect, the present disclosure provides methods of treating or prevent a disease or disorder comprising administering to a patient in need thereof a compound or composition described herein in a therapeutically effective amount. In some embodiments, the disease or disorder is alcoholism. In some embodiments, the patient is a mammal such as a human. In some embodiments, the disease or disorder is associated with the δ opioid receptor. In some embodiments, the compound or composition results in greater modulation of δ opioid receptor compared to μ opioid receptor.

In another aspect, the present disclosure provides methods of reducing alcohol composition in a patient comprising administering to the patient a therapeutically effective amount of a compound or composition described herein. In some embodiments, the patient is a mammal such as a human. In some embodiments, the compound or composition is associated with the δ opioid receptor. In some embodiments, the compound or composition results in greater modulation of δ opioid receptor compared to μ opioid receptor.

In still another aspect, the present disclosure provides methods of modulating the activity of a δ opioid receptor comprising contacting the δ opioid receptor with a compound or composition described herein. In some embodiments, the methods are performed in vitro. In other embodiments, the methods are performed in vivo. In other embodiments, the methods are performed ex vivo. In some embodiments, the compound or composition results in greater modulation of δ opioid receptor compared to μ opioid receptor.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C show kratom alkaloids reduce alcohol intake. Systemic (i.p.) injection of paynantheine (FIG. 1A), speciogynine (FIG. 1B), and 7-hydroxymitragynine (7-OH-mit) (FIG. 1C), on modified 2-bottle choice drinking behaviour in male C57BL/6 mice. Statistical significance was calculated with an unpaired, two-tailed t-test and is expressed as * when p<0.05

FIG. 2A-2C show paynantheine and speciogynine analog structures. Structures of naturally occurring kratom alkaloids paynantheine and speciogynine were used as scaffolds for analog synthesis. Analogs with pseudo-indoxyl (PI) rearrangements or hydroxyl group additions were made for both compounds, and a naturally occurring minor kratom alkaloid and speciogynine isomer, speciociliatine, was also synthesized for testing. (FIG. 2A) Chemical structures of selected indole based kratom alkaloids; (FIG. 2B) Synthesis of 7-hydroxypaynantheine (7) and paynantheine pseudoindoxyl (8); (FIG. 2C) Synthesis of 7-hydroxyspeciogynine (9) and speciogynine pseudoindoxyl (10).

FIG. 3A-3I show pharmacological characterization of kratom analogs at opioid receptors. Kratom alkaloid derivatives speciociliatine (SPECIO), speciogynine pseudo indoxyl (SPG PI), paynantheine pseudo indoxyl (PAYN PI), 7-hydroxy speciogynine (7OH SPG), and 7-hydroxy paynantheine (7OH PAYN) were characterized for binding affinity using [3H]DAMGO, [3H]DPDPE, [3H]U69,593 (FIG. 3A, FIG. 3B, FIG. 3C), inhibition of forskolin-induced cAMP in a Glo-sensor assay in transfected HEK-293 cells (FIG. 3D, FIG. 3E, FIG. 3F) and the ability of the alkaloids to recruit β-arrestin 2 in a PathHunter assay. (FIG. 3G, FIG. 3H, FIG. 3I) at μOR (FIG. 3A, FIG. 3D, FIG. 3G), δOR (FIG. 3B, FIG. 3E, FIG. 3H), and κOR (FIG. 3C, FIG. 3F, FIG. 3I). All curves are representative of the averaged values from a minimum of 3 independent assays.

FIGS. 4A-4G show kratom analogs decrease voluntary ethanol consumption in SOP-dependent mechanism. Kratom analogs 7-hydroxyspeciogynine (7OH SPG), 7-hydroxypaynantheine (7OH PAYN), and speciociliatine (SPECIO) are compared to kratom alkaloids (dashed lines; 7-hydroxymitragynine (7OH MITRA), paynantheine (PAYN), and speciogynine (SPG) for inhibition of forskolin-induced cAMP in a Glo-sensor assay in transfected HEK-293 cells at δOP (FIG. 4A) and μOP (FIG. 4B). Following three weeks of exposure to a voluntary two-bottle choice (10% alcohol vs. water), limited access, drinking-in-the-dark protocol, male and female C57Bl/6 wild-type mice and male δOP KO mice were injected with kratom analogs (s.c.) to address changes in volitional alcohol consumption (C-G). (FIG. 4C) In WT male mice, 7-hydroxypaynantheine (n=8) significantly decreased ethanol consumption at a 30 mg·kg⁻¹ dose but not a 10 mg·kg⁻¹ dose, and in (FIG. 4D) 7-hydroxyspeciogynine (n=12 male, n=9 female) dose-dependently decreased ethanol consumption at a 3 and 10 mg·kg⁻¹ dose. (FIG. 4E) In male, δOP KO mice (n=9), alcohol consumption was not significantly altered by a 3 or 10 mg·kg⁻¹ dose of 7-hydroxyspeciogynine, a 30 mg·kg⁻¹ dose of 7-hydroxypaynantheine, or a 10 mg·kg⁻¹ dose of paynantheine. (FIG. 4F) In WT male mice, speciociliatine decreased ethanol consumption at a 30 mg·kg⁻¹ dose (i.p.). (FIG. 4G) In a rotarod assessment of motor incoordination in WT and δOP KO mice (n=8 and n=7, respectively), a 30 mg·kg⁻¹ dose of speciociliatine (i.p.) significantly decreased time spent on the rod at 5, 15, 30, and 60 minutes post-injection compared to baseline (baseline represented at time=0 and the dotted line at y=100); significance for WT mice and δOP KO mice is denoted with stars and carats, respectively. Two bottle choice paradigms were analyzed using repeated measures 1-way ANOVA with Dunnett's multiple comparisons or with a mixed model with Dunnett's multiple comparisons for the combined male and female data. Thresholds for statistical significance: * or \hat{ } p<0.05, ** or \hat{ }\hat{ } p<0.01, *** p<0.001.

FIGS. 5A-5D show side effect profile of 10 mg·kg⁻¹ 7-hydroxyspeciogynine (FIG. 5A) In a 10-day conditioned place preference (CPP) paradigm, the rewarding effects of 7-hydroxyspeciogynine (s.c.) were evaluated in male, WT mice (n=8). (FIG. 5B) Locomotor data was extracted from the CPP experiment in (FIG. 5A) and averaged across all vehicle/drug treatment days (n=7). (FIG. 5C) 7-hydroxyspeciogynine was tested for agonist, analgesic properties in male mice via the tail flick thermal nociception assay (n=10). In the same paradigm, antagonistic effects were evaluated after administering 7-hydroxypeciogynine, followed by morphine (6 mg·kg⁻¹, s.c.) 10 minutes later (n=6) and were compared to vehicle plus morphine administration (n=5). (FIG. 5D) The highest racine score collected every 3 minutes for 30 minutes following administration of 7-hydroxyspeciogynine was evaluated for 30 minutes after drug administration (n=9). For the CPP and locomotor experiments, statistical significance was calculated with paired, two-tailed tests. For the locomotor experiment, one mouse was removed after being identified as an outlier with the Grubb's test. Nociception data is expressed as maximum possible effect (% MPE) normalized to a saline baseline (treatment—saline baseline). Statistical significance for agonist nociception experiments was calculated with paired, two-tailed tests between vehicle and drug dose. Statistical significance for antagonist nociception experiments was calculated with an unpaired t-test with Welch's correction between the treatment groups.

FIGS. 6A-6C show percent decrease in ethanol consumption by kratom analogs 2-bottle choice ethanol consumption data from FIG. 5 is revisualized as percent decreases in ethanol consumption for 7-hydroxyspeciogynine (7OH SPG), 7-hydroxypaynantheine (7OH PAYN), and speciociliatine (SPECIO). (FIG. 6A) In WT male and female mice (n=12 male, n=9 female), 7-hydroxyspeciogynine significantly decreased the percent ethanol consumption at a 3 and 10 mg·kg⁻¹ dose. (FIG. 6B) In WT male mice (n=8), 7-hydroxypaynanthiene decreased the percent ethanol consumption at a 30 but not 10 mg·kg⁻¹. (FIG. 6C) In WT male mice (n=11), speciociliatine decreased ethanol consumption at a 30 mg·kg⁻¹ dose. Thresholds for statistical significance: * p<0.05, ** p<0.01, **** p<0.0001.

### DESCRIPTION OF ILLUSTRATIVE EMBODIMENTS

Described herein are a series of compounds that show an improved pharmaceutical profile with respects to reduced μOP potency. Without wishing to be bound by any theory, it is believed that these compounds may decrease ethanol consumption via a δOP-dependent mechanism. In particular, these compounds may show reduced side effects such as reduced reward effects or may be less addictive than other opioids currently in clinical use. These and other aspects of the present disclosure are described in the claims and the following sections.

### I. Alcoholism and Opioid Receptors

A. Alcoholism and Alcohol Related Disorders

Drinking alcohol (ethanol or ethyl alcohol) is a learned response, reinforced largely by the rewarding effects of alcohol in the central nervous system, the euphoria from lower, stimulatory doses of ethanol. A person with an alcohol use disorder, colloquially referred to as an alcoholic, through an interplay of genetic and environmental factors, has had the alcohol-drinking response reinforced so often and so well that it becomes too strong for the individual to continue functioning properly in society. The strong alcohol-drinking response, i.e., the drive for alcohol, then dominates the person's behavior and life. The Diagnostic and Statistical Manual of Mental Disorders (DSM) 5^{th} edition provides guidance for the diagnosis of alcohol use disorder if a person meets 2 or more of 11 criteria during a 12 month period.

Alcoholism, or alcohol use disorder, is the most expensive health problem in many countries. Several treatment methods have been developed. According to Kranzler, despite the developments in treating alcoholism, such basic issues as the optimum dosing strategy and duration of treatment for existing therapies are not known (Kranzler, 2000). Some methods, such as counseling and Alcoholics Anonymous (AA), are aimed at increasing the alcoholic's ability or willpower to withstand the drive for alcohol. The drive, however, is not weakened and the patient is told that he will remain an alcoholic, that is, a person with an overly strong alcohol-drinking response, for the rest of his life. These methods succeed in some alcoholics, but in most cases eventually comes the time when a momentary decrease in willpower causes a resumption of alcohol drinking and alcohol abuse. These methods are not very successful because they do not effectively weaken the alcoholic's alcohol-drinking response.

Other treatments use punishment of various sorts (e.g., electric shock, disulfiram reactions, etc.) to try to stop alcohol drinking. Punishment is, however, a poor method for changing behavior and has many limitations. In particular, it is ineffective when positive reinforcement is still being received for the same response that is punished. Since the treatments that punish alcohol drinking do not block the positive reinforcement of the same response coming from alcohol in the brain, they should not be expected to be very effective.

In the FDA approved methods of treating an alcohol use disorder, the alcohol-drinking response is extinguished by administering an opioid antagonist, such as naltrexone, in conjunction with alcohol. Extinction consists of having the response emitted repeatedly in the absence of positive reinforcement. Much of the positive reinforcement for alcohol drinking is internal, from the rewarding effects of alcohol in the brain. U.S. Pat. No. 4,882,335 discloses a method for treating alcoholism in which the learned response of alcohol drinking is extinguished by being emitted while the reinforcement from alcohol in the brain is blocked with an opiate antagonist. In this extinction method, an opiate antagonist is administered to a subject suffering from alcoholism in a daily dosage Sufficient to block the stimulatory effect of alcohol and, while the amount of antagonist in the subject's body is sufficient to block the stimulatory effect of alcohol, the subject is made to drink an alcoholic beverage.

Furthermore, the desire to drink and consume alcohol appears to be associated with one or more opioid receptors such as the mu and delta opioid receptors. Therefore, these receptors may play a role in numerous alcohol abuse related conditions such as alcohol abuse, alcohol addiction, alcohol craving (including, but not limited to post-deprivation craving, post-withdrawal craving, relapse craving and binge craving), alcohol dependency, alcohol withdrawal, and related disorders. It is believed that compounds such as those described herein may be used to treat one or more of these conditions.

B. Opioid Receptors

Opioid receptors comprise a family of cell surface proteins, which control a range of biological responses, including pain perception, modulation of affective behavior and motor control, autonomic nervous system regulation and neuroendocrinologic function. There are three major classes of opioid receptors in the CNS, designated mu, kappa and delta, which differ in their affinity for various opioid ligands and in their cellular distribution. The different classes of opioid receptors are believed to serve different physiologic functions (Olson et al., 1989; Lutz and Pfister, 1992; and Simon et al., 1991; and Faouzi et al., 2020) Opiates, such as morphine, produces analgesia primarily through the mu-opioid receptor. However, among the opioid receptors, there is substantial overlap of function as well as of cellular distribution.

The mu-opioid receptor mediates the actions of morphine and morphine-like opioids, including most clinical analgesics. In addition to morphine, several highly selective agonists have been developed for mu-opioid receptors, including [D-Ala²,MePhe⁴,Gly(ol)⁵] enkephalin (DAMGO), levorphanol, etorphine, fentanyl, sufentanil, bremazocine and methadone. Mu-opioid receptor antagonists include naloxone, naltrexone, D-Phe-Cys-Try-D-Trp-Orn-Thr-Pen-Thr-NH₂ (CTOP), diprenorphine, β-funaltrexamine, naloxonazine, nalorphine, nalbuphine, and naloxone benzoylhydrazone. Differential sensitivity to antagonists, such as naloxonazine, indicates the pharmacologic distinctions between the mu-opioid receptor subtypes, mu₁, and mu₂. Several of the endogenous opioid peptides also interact with mu-opioid receptors.

There are three known kappa-opioid receptor subtypes, designated kappa₁, kappa₂ and kappa₃. Each kappa-opioid receptor subtype possesses distinct pharmacologic properties. For example, kappa₁-opioid receptors produce analgesia spinally and kappa₃-opioid receptors relieve pain through supraspinal mechanisms. In addition, the kappa₁-opioid receptor selectively binds to the agonist U50,488. Additional agonists of the kappa₁-opioid receptor include etorphine; sufentanil; butorphanol; β-funaltrexamine; nalphorine; pentazocine; nalbuphine; bremazocine; ethylketocyclazocine; U50,488; U69,593; spiradoline; and nor-binaltorphimine. Agonists of the kappa₃-opioid receptor include etorphine; levorphanol; DAMGO; nalphorine; nalbuphine; naloxone benzoylhydrazone; bremazocine; and ethylketocyclazocine. Effects of agonists on the kappa₁-opioidreceptors are reversed by a number of antagonists, including buprenorphine, naloxone, naltrexone, diprenorphine, naloxonazine, naloxone benzoylhydrazone, naltrindole and nor-binaltorphimine. Antagonists of the kappa₃-opioid receptors include naloxone, naltrexone and diprenorphine.

The delta-opioid receptors are divided into two subclasses, delta₁ and delta₂. The delta opioid receptors modulate analgesia through both spinal and supraspinal pathways. The two subclasses were proposed based on their differential sensitivity to blockade by several novel antagonists (Portoghese et al., 1992; Sofuoglu et al., 1991). The agonists [D-Pro²,Glu⁴] deltorphin and [D-Ser²,Leu⁵] enkephalin-Thr⁶ (DSLET) preferentially bind to the delta₂ receptors, whereas [D-Pen²,D-Pen⁵] enkephalin (DPDPE) has a higher affinity for delta₁ receptors.

There are three distinct families of endogenous opioid peptides, the enkephalins, endorphins and dynorphins. Each such peptide is derived from a distinct precursor polypeptide. Mu-opioid receptors have a high affinity for the enkephalins as well as β-endorphin and dynorphin A. The enkephalins are also endogenous ligands for the delta receptors, along with dynorphin A and dynorphin B. The kappa₁-opioid receptor endogenous opioid peptide agonists include dynorphin A, dynorphin B and α-neoendorphin. See Reisine and Pasternak (1996).

Members of each known class of opioid receptor have been cloned from human cDNA and their predicted amino acid sequences have been determined (Yasuda et al., 1993; Chen et al., 1993) The opioid receptors belong to a class of transmembrane spanning receptors known as G-protein coupled receptors. G-proteins consist of three tightly associated subunits, alpha, beta and gamma (1:1:1) in order of decreasing mass. Signal amplification results from the ability of a single receptor to activate many G-protein molecules, and from the stimulation by G-α-GTP of many catalytic cycles of the effector. Most opioid receptor-mediated functions appear to be mediated through G-protein interactions (Standifer and Pasternak, 1997) Antisense oligodeoxynucleotides directed against various G-protein alpha subunits were shown to differentially block the analgesic actions of the mu-, delta-, and kappa-opioid agonists in mice (Standifer et al., 1996)

### II. Compounds of the Present Disclosure

The compounds of the present disclosure are shown, for example, above, in the summary of the invention section, and in the claims below. They may be made using standard methods that can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the kratom alkaloid derivatives of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the kratom alkaloid derivatives of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise. In particular, the compounds described herein may have a better pharmacological profile in that the show reduced activation of beta-arrestin.

Kratom alkaloid derivatives of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present opiate compounds may contain two or more atoms which have a defined stereochemical orientation.

Chemical formulas used to represent kratom alkaloid derivatives of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the kratom alkaloid derivatives of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, kratom alkaloid derivatives of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### III. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a opiate compounds disclosed herein formulated with one or more excipients and/or drug carriers appropriate to the indicated route of administration. In some embodiments, the kratom alkaloid derivatives disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the kratom alkaloid derivatives disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The kratom alkaloid derivatives disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The kratom alkaloid derivatives disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a kratom alkaloid derivative disclosed herein or composition comprising a kratom alkaloid derivative disclosed herein administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 98 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### IV. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof, “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “ [symbol] ” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, the formula

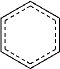

covers, for example,

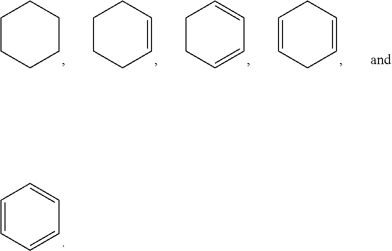

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

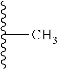

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

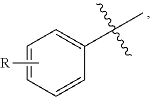

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

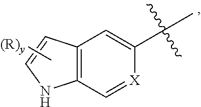

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

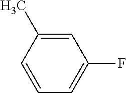

is also taken to refer to

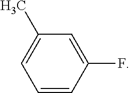

Aromatic compounds may also be depicted using a circle to represent the delocalized nature of the electrons in the fully conjugated cyclic π system, two non-limiting examples of which are shown below:

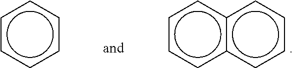

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

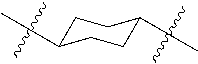 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

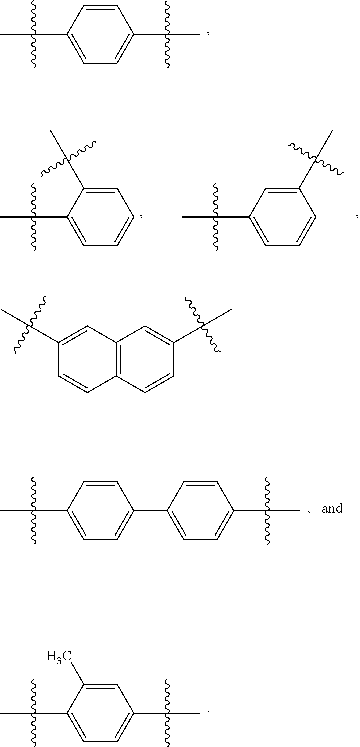

An “arene” refers to the class of compounds having the formula H—R, wherein R is aryl as that term is defined above. Benzene and toluene are non-limiting examples of arenes.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings may be fused, bridged, or spirocyclic. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, tetrahydropyridinyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, horse, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity with in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods and Materials

### A. Materials

Kratom “Red Indonesian Micro Powder” was purchased from Moon Kratom (Austin, Tex.). Leu-enkephalin, forskolin, and morphine sulfate pentahydrate were purchased from Sigma Aldrich (St. Louis, Mo., USA). (2S)-2-[[2-[[(2R)-2-[[(2S)-2-Amino-3-(4-hydroxyphenyl)propanoyl]amino]propanoyl] amino]acetyl]-methylamino]-N-(2-hydroxyethyl)-3-phenylpropanamide (DAMGO), 2-(3,4-dichlorophenyl)-N-methyl-N-[(1R,2R)-2-pyrrolidin-1-ylcyclohexyl]acetamide (U50,488), and naloxone hydrochloride were purchased from Tocris Bioscience (Bio-techne Corporation, Minneapolis, Minn., USA). [3H]DAMGO (53.7 Ci/mmol, lot #2376538; 51.7 Ci/mmol, lot #2815607), [3H]U69,593 (60 Ci/mmol, lot #2367921 and lot #2644168; 49.2 Ci/mmol, lot #2791786), [3H]DPDPE (49.2 CI/mmol, lot #2573313 and lot #2726659; 48.6 Ci/mmol, lot #2826289) were purchased from Perkin Elmer (Waltham, Mass., USA). For in vivo experiments, morphine and naloxone were prepared in a saline vehicle. Kratom derived analogs were dissolved in a 1:1:8 ethanol:cremophor:saline vehicle for all behavioral experiments. For the 2-bottle choice experiment in δOP KO mice, paynantheine was prepared in the same 1:1:8 ethanol:cremophor:saline vehicle. For all other experiments paynantheine and speciociliatine were dissolved in a slightly acidic saline solution that was adjusted to a pH of 6-7 before administration.

### B. Chemistry

i. General

All chemicals were purchased from Sigma-Aldrich Chemicals and used without further purification. Reactions were carried out in flame-dried reaction flasks under argon. Reaction mixtures were purified by silica flash chromatography on E. Merck 230-400 mesh silica gel 60 using a Teledyne ISCO CombiFlash Rf instrument with UV detection at 280 and 254 nm. RediSep Rf silica gel normal phase columns were used. The yields reported are isolated yields. NMR spectra were recorded on a Varian 400/500 MHz NMR spectrometer. NMR spectra were processed with MestReNova software. The chemical shifts were reported as δ ppm relative to TMS using residual solvent peak as the reference unless otherwise noted (CDCl3 1H: 7.26, 13C: 77.3). Peak multiplicity is reported as follows: s, singlet; d, doublet; t, triplet; q, quartet; m, multiplet. Coupling constants (J) are expressed in Hz. High resolution mass spectra were obtained on a Bruker Daltonics 10 Tesla Apex Qe Fourier Transform Ion Cyclotron Resonance-Mass Spectrometer by electrospray ionization (ESI). Accurate masses are reported for the molecular ion [M+Na]+.

ii. Isolation of Mitragynine from Mitragyna speciosa (Kratom)

Mitragynine was extracted from the powdered leaves by following previously reported methods (Gutridge et al., 2020; Váradi et al., 2016). Kratom powder (500 g) was heated to reflux in MeOH 700 mL for 40 min. The suspension was filtered and the methanolic extraction process was repeated (3×500 mL). The solvent of combined methanolic extract was removed under reduced pressure and the content was dried using high vacuum. The dry residue was resuspended in 20% acetic acid solution (1 L) and washed with petroleum ether (4×500 mL). The aqueous layer was then cooled on ice bath and basified (pH ˜9) with aqueous NaOH solution (3.5M. ˜1 L) slowly. Alkaloids were extracted in DCM (4×400 mL) from the aqueous layer. The combined DCM layer was washed with brine 300 mL and dried over anhydrous Na₂SO₄ and filtered. The solvent was removed under reduced pressure, and the residue was dried under high vacuum to obtain kratom extract (9.8 g). Then, this crude kratom extract was subjected to silica gel column chromatography; using 0-15% MeOH in dichloromethane to isolate mitragynine (4.7 g); paynantheine (568 mg), speciogynine (343 mg), and speciociliatine (754 mg) along with some minor alkaloids.

iii. 7-Hydroxypaynantheine (7OH Pay/7)

Paynantheine (100 mg, 0.25 mmol) was dissolved in acetonitrile (7 mL), then water (2 mL) was added. The resulting suspension was cooled to 0° C., and PIFA (108 mg, 1.1 equiv) dissolved in acetonitrile (1.1 mL) was added slowly over the course of several minutes. The reaction mixture was stirred at 0° C. for 45 minutes. Then, saturated aqueous NaHCO₃ solution was added, and the mixture extracted with EtOAc (3×15 mL). The organic phase was washed with brine (20 mL) and dried over anhydrous Na₂SO₄. The solvent was removed under reduced pressure. The residue was purified on a silica column using 10-75% EtOAc in hexanes as eluent. The fractions containing the product were evaporated to yield 42 mg (40%) of 9 as a light magenta amorphous powder. ¹H δ (400 MHz, ppm): 7.31 (1H, s, 17); 7.29 (1H, t, 3J=7.7 Hz, 11); 7.19 (1H, t, 3J=7.7 Hz, 12); 6.74 (1H, d, 3J=7.7 Hz, 10); 5.57 (1H, ddd, 3J=18.0, 10.3, 7.2 Hz, 19); 4.99 (1H, dd, 3J=18.0, 2J=1.5 Hz, 18 trans); 4.94 (1H, dd, 3J=10.3, 2J=1.5 Hz, 18 cis); 3.86 (3H, s, 9-OMe); 3.79 (3H, s, 17-OMe); 3.68 (3H, s, 16-COOMe); 3.46 (1H, s, 7-OH); 3.23 (1H, m, 3); 3.03 (1H, m, 21/1); 3.01 (1H, m, 20); 2.85 (1H, m, 5/2); 2.73 (1H, m, 5/1); 2.72 (1H, m, 15); 2.66 (1H, m, 6/1); 2.39 (1H, m, 14/1); 2.30 (1H, m, 21/2); 2.05 (1H, m, 14/2); 1.70 (1H, m, 6/2); 13C δ (100 MHz, ppm): 183.5 (2); 168.8 (16-CO); 159.8 (17); 155.9 (9); 154.9 (13); 139.3 (19); 131.0 (11); 126.4 (8); 115.4 (18); 114.3 (12); 111.4 (16); 109.1 (10); 81.0 (7); 61.6 (21); 61.5 (17-OMe); 60.2 (3); 55.5 (9-OMe); 51.2 (16-COOMe); 49.8 (5); 42.8 (20); 38.2 (15); 35.9 (6); 30.4 (14). Relative configuration was determined based on the NOE cross peaks between the following ¹H nuclei: 3-5/2; 3-14/2; 3-21/2; 3-5/2; 15-19; 19-21/2 (/1 always indicates the hydrogen pointing towards the reader from the paper; /2 indicate the hydrogen pointing behind the plain of the paper). HRMS (ESI-TOF) m/z: [M+Na]⁺ Calcd for C₂₃H₂₈N₂NaO₅ 435.189043; found. 435.189116.

iv. Paynantheine Pseudoindoxyl (Pay PI/8)

7-hydroxypaynantheine (9, 40 mg, 0.1 mmol) was dissolved in dry toluene (1.5 mL), and Zn(OTf)₂ (70 mg, 2 equiv) was added. The reaction mixture was stirred in a sealed tube for 30 minutes at 115° C. To the cooled mixture were added 2 mL sat. aqueous NaHCO₃ solution and water (5 mL) and the organics were extracted with EtOAc (10 mL). The organic layer was rinsed with brine (10 mL) and dried over anhydrous Na₂SO₄. After evaporation of the solvent under reduced pressure, the residue was purified by flash column chromatography on silica (gradient: 40-75% EtOAc in hexanes) to yield 15 mg (38%) of product as a light yellow gum. ¹H δ (400 MHz, ppm): 7.32 (1H, t, 3J=8.2 Hz, 11); 7.18 (1H, s, 16); 6.37 (1H, d, 3J=8.2 Hz, 12); 6.13 (1H, d, 3J=8.2 Hz, 10); 5.49 (1H, ddd, 3J=18.2, 10.3, 7.4 Hz, 19); 5.25 (1H, br s, 1); 4.95 (1H, d, 3J=18.2, 18 trans); 4.9 (1H, d, 3J=10.3, 18 cis); 3.89 (3H, s, 9-OCH3); 3.73 (3H, s, 17-OCH3); 3.62 (3H, s, 16-COOCH3); 3.23 (1H, m, 5/1); 3.11 (1H, m, 21/1); 2.87 (1H, m, 20); 2.49 (1H, m, 15); 2.39 (1H, m, 5/2); 2.39 (1H, m, 6/2); 2.34 (1H, m, 3); 1.98 (1H, m, 21/2); 1.94 (1H, m, 6/1); 1.79 (1H, br q 3J=11.3 Hz, 14/1); 1.26 (1H, br d, 3J=11.3 Hz, 14/2). 13C δ (100 MHz, ppm): 199.8 (7); 168.2 (16-C═O); 162.1 (13); 159.7 (17); 158.7 (9); 139.5 (19); 139 (11); 115.6 (18); 111.9 (16); 109.5 (8); 104 (12); 99.2 (10); 74.7 (2); 72.4 (3); 61.5 (17-O—CH3); 58.8 (21); 55.8 (9-OCH3); 53.2 (5); 51.1 (COO—CH3); 42.3 (20); 36.9 (15); 35.3 (6); 28.3 (14). Relative configuration was determined based on the NOE cross peaks between the following 1H nuclei: 1-6/1; 3-14/2; 1-14/1; 14/1-20; 15-19; 19-21/2. HRMS (ESI-TOF) m/z: [M+Na]⁺ Calcd for C₂₃H₂₈N₂NaO₅ 435.189043; found. 435.189219.

v. 7-hydroxyspeciogynine (7OH Spg/9)

Speciogynine (200 mg, 0.5 mmol) was dissolved in acetonitrile (15 mL), then water (5 mL) was added. The resulting suspension was cooled to 0° C., and PIFA (216 mg, 1.1 equiv) dissolved in acetonitrile (2.2 mL) was added slowly over the course of several minutes. The reaction mixture was stirred at 0° C. for one hour. Then, saturated aqueous NaHCO₃ solution was added, and the mixture extracted with EtOAc (3×40 mL). The organic phase was washed with brine (30 mL) and dried over anhydrous Na₂SO₄. The solvent was removed under reduced pressure. The residue was redissolved in DCM and was purified using silica column chromatography 10-75% EtOAc in hexanes. The fractions containing the product were evaporated to yield 107 mg (57%) of 9 as a light brown amorphous powder. ¹H NMR (400 MHz, chloroform-d) δ 7.36-7.29 (m, 1H), 7.26 (dd, J=8.8, 7.2 Hz, 1H), 7.17 (d, J=7.7 Hz, 1H), 6.71 (d, J=8.3 Hz, 1H), 3.84 (s, 3H), 3.75 (s, 3H), 3.66 (s, 3H), 3.21-3.08 (m, 2H), 2.82 (t, J=12.3 Hz, 1H), 2.77-2.69 (m, 1H), 2.64 (d, J=14.4 Hz, 1H), 2.54 (t, J=11.2 Hz, 1H), 2.30 (d, J=11.9 Hz, 1H), 2.17 (t, J=10.5 Hz, 1H), 2.06 (t, J=11.2 Hz, 2H), 1.80 (s, 1H), 1.69 (td, J=13.5, 4.5 Hz, 1H), 1.40 (s, 1H), 1.02 (d, J=17.1 Hz, 1H), 0.82 (t, J=7.4 Hz, 3H). 13C NMR (100 MHz, chloroform-d) δ 183.9, 169.61, 160.10, 156.07, 155.15, 131.15, 126.52, 114.42, 111.44, 109.18, 81.16, 61.98, 61.49, 61.52, 55.66, 51.64, 50.21, 39.54, 38.87, 36.13, 24.49, 11.56, 11.29. HRMS (ESI-TOF) m/z: [M+Na]⁺ Calcd for C₂₃H₃₀N₂NaO₅ 437.204693; found. 437.204951.

vi. Speciogynine Pseudoindoxyl (Spg PI/10)

7-hydroxyspeciogynine (9, 200 mg, 0.48 mmol) was dissolved in dry toluene (6 mL), and Zn(OTf)₂ (350 mg, 2 equiv) was added. The reaction was stirred in a sealed tube for 2 h at 100° C. To the cooled mixture were added 10 mL sat. aqueous NaHCO₃ solution and water (20 mL). Extracted with EtOAc (30 mL). The organic layer was rinsed with brine (20 mL) and dried over anhydrous Na₂SO₄. After evaporation of the solvent under reduced pressure, the residue was redissolved in DCM and purified by flash column chromatography (gradient: 40-75% EtOAc in hexanes) to yield 78 mg (39%) of 10 as a light yellow amorphous powder. ¹H NMR (500 MHz, chloroform-d) 7.31 (1H, t, 3J=8.2 Hz, 11), 7.23 (1H, s, 17), 6.36 (1H, d, 3J=8.2 Hz, 12), 6.12 (1H, d, 3J=8.2 Hz, 10), 5.34 (1H, br s, 1), 3.89 (3H, s, 9-OMe), 3.72 (3H, s, 17-OMe), 3.62 (3H, s, 16-COOMe), 3.25-3.23 (1H, m, 21/1), 3.22-3.21 (1H, m, 5/1), 2.37-2.35 (2H, m, 5/2; 6/2), 2.33-2.31 (1H, m, 15), 2.29-2.28 (1H, m, 3), 2.08-2.04 (1H, m, 20), 1.94-1.90 (1H, m, 6/1), 1.81-1.77 (1H, m, 14/1), 1.75-1.73 (1H, m, 21/2), 1.34-1.30 (1H, br m, 19/1), 1.18-1.15 (1H, m, 14/2), 0.95-0.92 (1H, br m, 19/2), 0.79 (3H, br, 18). 13C NMR (100 MHz, chloroform-d) 200.18 (7), 168.02 (16-CO), 162.25 (13), 160.27 (17), 158.83, (9), 139.17 (11), 112.22 (16), 109.5 (8), 104.26 (12), 99.17 (10), 74.94 (2), 72.94 (3), 61.51 (17-OMe), 58.42 (21), 55.99 (9-OMe), 53.57 (5), 51.07 (16-COOMe), 38.15 (20), 37.50 (15), 35.48 (6), 28.95 (4), 24.46 (9), 11.35 (18). Relative configuration was determined based on the NOE cross peaks between the following 1H nuclei: 1-6/1; 1-14/1; 15-19; 19-21/2. (/1 always indicates the hydrogen pointing towards the reader from the paper; /2 indicate the hydrogen pointing behind the plain of the paper). HRMS (ESI-TOF) m/z: [M+Na]⁺ Calcd for C₂₃H₃₀N₂NaO₅ 437.204693; found. 437.204760.

### C. Cellular Assays

Membrane Isolation and Competitive Radioligand Binding Assay: Membrane isolation and subsequent binding assays were completed as described previously using membranes stably expressing the μOP, δOP, or κOP were isolated from CHO (μOP, δOP) or U2OS cells (κOP) (DiscoverX) and using OP specific radiolabels [³H]DAMGO, [³H]DPDPE and [³H]U69,593 (Cassell et al., 2019; Creed et al., 2020). GloSensor cAMP Inhibition Assay: cAMP inhibition assays were performed in HEK cells transiently transfected with pGloSensor22F and either expressing FLAG-mouse δOP, HA-mouse μOP, or FLAG-mouse κOP as previously described (Chiang et al., 2016). PathHunter β-arrestin2 Recruitment Assay: β-arrestin recruitment assays were performed in PathHunter cells stably expressing the μOP, δOP, or κOP and β-arrestin 2 as previously described (Chiang et al., 2016; Chakraborty et al., 2021).

### D. Animals

The animal protocol (#1305000864) describing the care and use of experimental animals was approved by the Purdue University Institutional Animal Care and Use Committee (www.purdue.edu/research/regulatory-affairs/animal-research/staff.php). Animal studies were carried out in accordance with the ARRIVE guidelines (Kilkenny et al., 2010) and recommendations made by the British Journal of Pharmacology as well as recommendations of the National Institutes of Health Guide for the Care and Use of Laboratory Animals. Wildtype C57Bl/6N mice (108 male, 12 female; 6-7-weeks old) were purchased from Envigo (Indianapolis, Ind.) and were acclimated to the facility and to handling and injections for 1 week prior to any experimental procedures. δOP KO mice (17 male, 10-12 weeks old) with a C57Bl/6N background (recently re-derived) were bred in house and were similarly conditioned to handling and injections prior to experimentation. All mice were housed on a reverse 12-hour light (21:30-9:30)/12-hour dark cycle under controlled temperature (21-23° C.) with ad libitum food access. The only exception to this is mice used in the rotarod assay; these mice were housed in 12-hour light (6:00-18:00)/12-hour dark cycle. All experiments were conducted between 10:30-15:00, and all mice were habituated to the test room at least 30 minutes prior to experimentation. Rotarod, nociception, and seizure experiments were conducted in well-lit rooms whereas conditioned place preference, 2-bottle choice, and locomotor experiments were conducted in the dark. At a minimum, mice were given 2 days between experiments to recover from thermal stimuli. For the paynantheine agonist nociception assays, 10 male mice were exposed to two doses of paynantheine (10 and 30 mg·kg⁻¹, i.p.) For the paynantheine antagonist nociception assays, a separate group of 10 mice were exposed to 6 mg·kg⁻¹ morphine (s.c.) by itself then after treatment with 10 and 30 mg·kg⁻¹ paynantheine (i.p.) For acute and extended conditioned place preference experiments, separate groups of mice were used for each drug dose. A separate group of mice was used for the 7-hydroxymitragynine-block locomotor experiment with naloxone. For the 2-bottle choice alcohol consumption experiments with WT male and female mice, separate groups of mice were used to test increasing doses of each analog. For the 2-bottle choice experiments with δOP KO mice, the same group of mice was repeatedly tested with different drug treatments. Following a 3-week period of alcohol withdrawal, these δOP KO mice were used to examine seizure activity of paynantheine (30 mg·kg⁻¹, i.p.). After a week following the experiment, 5 wildtype male mice used in the naloxone-block locomotor experiments with 7-hydroxymitragynine were used to assess seizure activity of 30 mg·kg⁻¹ paynantheine (i.p.). Note that one δOP KO mouse died after experiencing severe, level 5-6 seizures following i.p. administration of 30 mg/kg speciociliatine in the rotarod assay, leading to an overall n=7 instead of n=8 for this genotype. All experimental procedures were approved by the Purdue Animal Care and Use Committee of Purdue University under protocols #1305000864 and #1605001408.

### E. Behavioral Assays

Tail Flick Thermal Nociception Assay: Antinociception via the tail flick assay was measured as previously described (van Rijn et al., 2012). Mice were first habituated to the handling restraint used during the experimentation. On subsequent test days, a radiant heat tail-flick instrument (Columbus Instruments, Columbus, Ohio, USA) was used to collect duplicate measurements by testing two different regions on the mouse's tail. The beam intensity was adjusted between each group of mice to elicit reproducible responses between 2-3 seconds (beam intensity of 7-9). For each test day, a baseline tail flick response was collected for each mouse and was used to calculate the testing cut-off time (cutoff time=three times the baseline response time). To test antinociception by drug agonism, a vehicle injection was next administered (i.p. or s.c.) and tail-flick responses were collected after 30 minutes. The drug was then administered (i.p. or s.c.) and tail flick responses were collected after 30 minutes. To test drug antagonism of morphine antinociception, a response to vehicle injections were similarly collected prior to drug administration with a first vehicle injection (i.p. or s.c.) at 0 minutes, followed by a second vehicle injection (s.c.) at 10 minutes before collecting tail flick responses at 30 minutes (twenty minutes after the second vehicle injection. The test compound was then administered (i.p. or s.c.), followed by 6 mg·kg⁻¹ morphine (s.c.) 10 minutes later. Tail-flick responses were collected 20 minutes following morphine administration. Data is represented as percent maximal possible effect (% MPE) and is calculates as % MPE=(treatment response time−baseline response time)/(cutoff time−baseline response time)*100. Data is normalized to vehicle treatment: drug treatment % MPE−saline treatment % MPE. Brief and Extended Conditioned Place Preference Paradigms: Mice were conditioned to drugs and vehicle as described previously in two-chamber conditioned place preference (CPP) boxes in a counterbalanced, unbiased approach (Gutridge et al., 2020; Váradi et al., 2015). Locomotor Evaluation: To assess drug-induced effects on ambulation for paynantheine and 7-hydroxyspeciogynine, locomotor information was extracted from the data generated in the CPP experiments. Distance traveled during each drug and vehicle conditioning session was pulled from the 30- or 40-minute conditioning session (extended or brief CPP, respectively) and all sessions per treatment were averaged for analysis. To assess drug-induced effects on ambulation for 7-hydroxymitragynine, locomotor activity was assessed in a 2-day protocol as previously described (Gutridge et al., 2020). Accelerating Rotarod Test: Mice were trained to walk on a rotarod apparatus (IITC, USA) with 1.25″ diameter drums on two days prior to drug testing. The rotarod started at 3 rpm and increased to 30 rpm over 300 seconds. A trial for a mouse ended when it fell and tripped the sensor, when it rode the rotarod for two consecutive revolutions. or after 300 seconds (the maximum trial time) (White et al., 2015). Mice received at least three minutes of rest between trials. On test day, baseline performance was assessed as the average latency to fall in three trials per mouse. Mice were then injected with 30 mg·kg⁻¹ speciociliatine (i.p.) and immediately tested for performance on the apparatus (this first data point represented as latency to fall at 5 minutes), and then tested again at 15, 30, 60, and 120 minutes post-injection. Each mouse's performance was normalized to its own baseline and reported as a percentage. Seizure Assay: To assess drug-induced seizurogenic activity, mice were placed in a clear plastic cylinder (25 cm diameter, 35 cm height) immediately following drug injection and their activity was recorded in a well-lit, quiet room using iSpy camera software (iSpyConnect.com). A recording time of 90 minutes was chosen for the tested compounds based on previous observations of seizures time lengths in experiments with 30 mg·kg⁻¹ paynantheine. If animals were not presenting with seizure activity after 30 minutes, the recording time was shortened accordingly. Seizure severity was scored based on the modified racine scale (half-scores allowed) in bins of 3-5 minutes. Onset to first seizure symptom, onset to highest racine score, and highest racine score were also assessed. Two-Bottle Choice Alcohol Paradigm: Mice were subject to a drinking in the dark (DID), limited access (four hours per day), 2-bottle choice (10% ethanol versus water) paradigm in which they were trained to consume alcohol voluntarily as previously described ( ). Mice reached stable alcohol consumption within three weeks of training, and after the third week, drug injections were administered prior to the daily drinking session on Friday. Drug effect on alcohol consumption was measured as the change in Friday's alcohol intake minus the average alcohol intake from the preceding Tuesday-Thursday of that week (g/kg).

### F. Data and Statistical Analysis

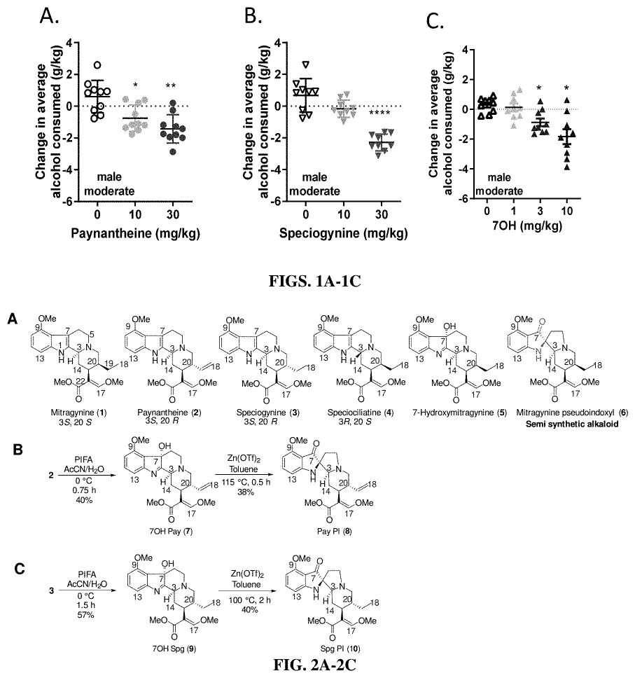

Data and statistical analysis comply with the recommendations on experimental design and analysis in pharmacology (Curtis et al., 2018). Data analysis was completed using GraphPad 9 (GraphPad Prism software, La Jolla, Calif.) and is presented as means±SEM. For findings from cellular assays, composite figures are shown consisting of an averaged curve from a minimum of three independent assays that were normalized to a positive control; best fit values in Table 1 were generated by GraphPad Prism from composite figures. For agonist antinociception assays, significance was calculated via a two-tailed, paired t-test to compare saline and drug treatment. For antagonist antinociception assays with three treatment groups in the same group of mice (FIG. 2D), data was analyzed via repeated measures (RM) one-way ANOVA with Dunnett's multiple comparisons to the morphine-only treatment group. For antagonist antinociception assays with two treatment groups in two different groups of mice (FIG. 6D), an unpaired t-test with Welch's correction was used to assess significance between the morphine-only group and the morphine plus “antagonist” group. All CPP data was analyzed with two-tailed, paired t-tests comparing time spent on the drug-paired side pre- and post-conditioning. For locomotor data in FIG. 1, an unpaired, two-tailed t-test was used. For locomotor data in FIG. 2G, statistical significance was obtained by a one-way ANOVA with Dunnett's multiple comparisons to VEH+VEH. For locomotor data in FIG. 6B, a two-tailed, paired t-test was used. For rotarod data, data for each tested time point was calculated as a percentage of the baseline, and thus statistical significance was calculated in a two-tailed, one sample t-test versus a hypothetical mean of 100 (baseline was 100%). Rotarod results between WT and δOP KO genotypes was compared with a mixed-effects model with fixed effects for timepoint, genotype, and timepoint x genotype. Seizure-like behavior between wildtype and δOP KO mice was compared with a two-tailed, unpaired t-test with Welch's correction on area-under the curve data generated from graphing the highest racine score per time bin over 90 minutes for each mouse. Results from 2-bottle choice alcohol consumption paradigms where more than one drug dose was tested were assessed for statistical significance using RM one-way ANOVA with Dunnett's multiple comparisons to the vehicle treated group. For alcohol consumption data where only one drug dose was tested, a paired, two-tailed t-test was used. For the alcohol consumption data for 7-hydroxyspeciogynine where male and female data was analyzed together, a mixed-effects model was used (due to missing values) with Dunnett's multiple comparisons to the vehicle-treated group.

### G. Nomenclature of Targets and Ligands

Key protein targets and ligands in this article are hyperlinked to corresponding entries in www.guidetophannacology.org, the common portal for data from the IUPHAR/BPS Guide to PHARMACOLOGY (Harding et al., 2018), and are permanently archived in the Concise Guide to PHARMACOLOGY 2019/20 (Alexander et al., 2019).

### Example 2: Results

TABLE 1
Kratom analogs characterization summary

| | Binding | | cAMP | | | β-arrestin 2 | |
|---|---|---|---|---|---|---|---|
| Compounds | pKᵢ | Kᵢ (μM) | pIC₅₀ | IC₅₀ (μM) | α | pEC₅₀ | α |
| μOP | | | | | | | |
| DAMGO | 9.6 ± 0.1 (1) | 0.00024 | 8.0 ± 0.1 (6) | 0.0099 | 100 | 6.6 ± 0.1 (6) | 100 |
| 7-OH MITRA | 7.7 ± 0.1 (6) | 0.0019 | *7.8 ± 0.1 (5) | 0.016 | 84 ± 3 | ND (3) | ND |
| SPG | 6.2 ± 0.1 (5) | 0.059 | *5.5 ± 0.1 (5) | 4.21 | 87 ± 6 | ND (3) | ND |
| PAYN | 6.3 ± 0.1 (4) | 0.052 | *5.4 ± 0.1 (5) | 4.08 | 100 ± 0 | ND (3) | ND |
| SPECIO | 7.1 ± 0.1 (3) | 0.086 | 6.4 ± 0.2 (5) | 0.43 | 38 ± 3 | ND (4) | ND |
| SPG PI | 7.1 ± 0.1 (3) | 0.077 | 6.6 ± 0.2 (5) | 0.23 | 58 ± 4 | ND (4) | ND |
| 7OH SPG | 7.7 ± 0.1 (3) | 0.021 | 6.2 ± 0.2 (6) | 2.40 | 66 ± 6 | ND (4) | ND |
| 70H PAYN | 5.2 ± 0.1 (3) | 6.15 | 4.7 ± 0.5 (5) | 21.8 | 80 ± 40 | ND (3) | ND |
| PAYN PI | 6.2 ± 0.1 (3) | 0.68 | 5.3 ± 0.2 (4) | 4.82 | 60 ± 6 | ND (3) | ND |
| δOP | | | | | | | |
| Leu-Enk | 9.2 ± 0.1 (3) | 0.00070 | 8.4 ± 0.1 (9) | 0.0042 | 100 | 7.4 ± 0.1 (7) | 100 |
| 7-OH MITRA | 6.7 ± 0.1 (4) | 0.019 | *5.7 ± 0.2 (8) | 0.96 | 80 ± 8 | 6.4 ± 0.3 (6) | 14 ± 1 |
| SPG | 5.1 ± 0.1 (6) | 5.34 | *5.0 ± 0.3 (5) | 12.4 | 94 ± 4 | ND (3) | ND |
| PAYN | 5.3 ± 0.1 (5) | 7.82 | *5.6 ± 0.2 (4) | 3.55 | 64 ± 13 | ND (3) | ND |
| SPECIO | 5.4 ± 0.1 (3) | 4.34 | ND (3) | ND | ND | ND (5) | ND |
| SPG PI | 6.0 ± 0.1 (3) | 0.94 | 5.1 ± 0.3 (4) | 8.53 | 80 ± 20 | ND (4) | ND |
| 7OH SPG | 6.3 ± 0.1 (3) | 0.46 | 5.6 ± 0.1 (6) | 2.27 | 76 ± 6 | ND (4) | ND |
| 70H PAYN | 4.9 ± 0.2 (4) | 12.7 | 5.2 ± 0.3 (5) | 5.74 | 70 ± 20 | ND (3) | ND |
| PAYN PI | 6.0 ± 0.1 (3) | 0.92 | ND (5) | ND | ND | ND (3) | ND |
| κOP | | | | | | | |
| U50,488 | 10.0 ± 0.2 (2) | 0.000099 | 8.5 ± 0.1 (5) | 0.0034 | 100 | 7.1 ± 0.1 (6) | 100 |
| 7-OH MITRA | 6.9 ± 0.1 (4) | 0.014 | *6.2 ± 0.3 (9) | 1.04 | 77 ± 5 | ND (4) | ND |
| SPG | 5.4 ± 0.1 (5) | 3.0 | *4.7 ± 0.3 (5) | 6.55 | 70 ± 20 | ND (4) | ND |
| PAYN | 5.5 ± 0.1 (5) | 4.0 | *5.3 ± 0.2 (4) | 7.43 | 95 ± 5 | ND (6) | ND |
| SPECIO | 6.2 ± 0.1 (4) | 0.59 | 5.6 ± 0.2 (4) | 2.50 | 60 ± 7 | ND (5) | ND |
| SPG PI | 6.1 ± 0.1 (3) | 0.75 | 4.7 ± 0.5 (4) | 20.6 | 80 ± 30 | ND (3) | ND |
| 7OH SPG | 5.8 ± 0.2 (3) | 1.63 | 5.1 ± 0.3 (3) | 7.71 | 80 ± 20 | ND (5) | ND |
| 70H PAYN | 5.1 ± 0.1 (3) | 7.46 | ND (3) | ND | ND | ND (3) | ND |
| PAYN PI | 5.9 ± 0.1 (4) | 1.31 | ND (3) | ND | ND | ND (3) | ND |

B. Kratom Analogs are OP Partial Agonists with Minimal β-Arrestin2 Recruitment.

In order to produce better lead candidates to treat alcohol use disorder that lack adverse locomotor and rewarding effects, kratom alkaloids or alkaloid derivatives with increased δOP affinity and potency, but with limited μOP potency were sought. To this end, paynantheine (2), speciogynine (3) and speciociliatine (4) were extracted from dry kratom powder using a modified protocol reported by Váradi et al. Paynantheine (2) was converted to 7-hydroxypaynantheine (7), FIG. 21B) using PIFA in acetonitrile and water. This 7-hydroxypaynantheine was next transformed to paynantheine pseudoindoxyl (8) using Zn(OTf)₂ in refluxing toluene. The same strategy to synthesize 7-hydroxyspeciogynine (9) and speciogynine pseudoindoxyl (10) as shown in FIG. 2C was used.

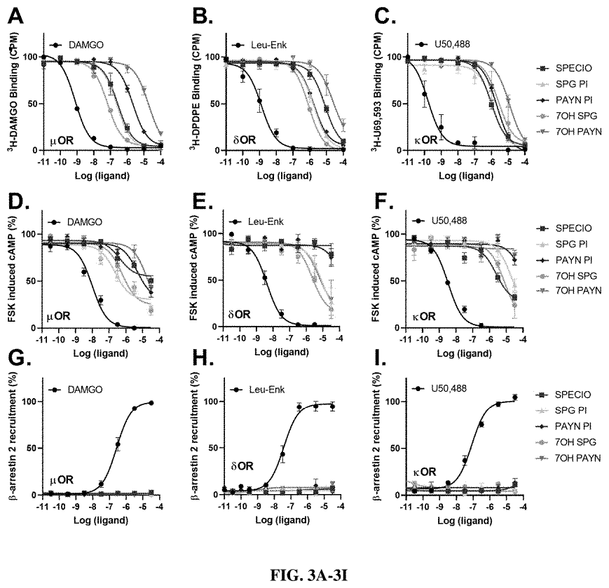

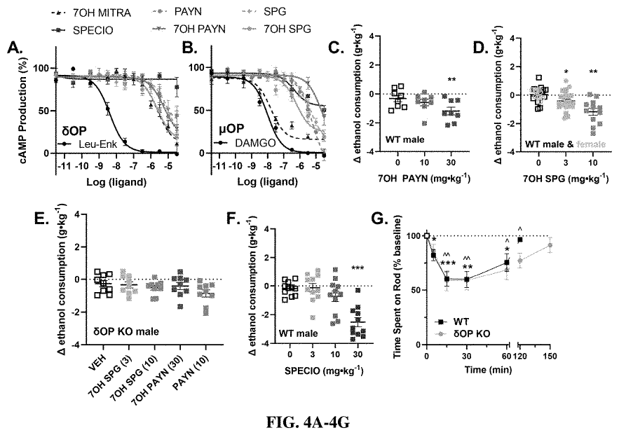

Affinity wise, the paynantheine analogs, especially the 7-hydroxyl analog, were noted to show weak μOP affinity, whereas 7-hydroxyspeciogynine displayed the strongest μOP affinity (FIG. 3A, Table 1). At the δOP, 7-hydroxyspeciogynine displayed improved binding relative to speciogynine which was on par with affinities for the two pseudoindoxyl analogs. 7-hydroxypaynantheine was a magnitude weaker in binding the δOP than 7-hydroxyspeciogynine; this same trend was apparent at the κOP (FIG. 3A-C, Table 1).

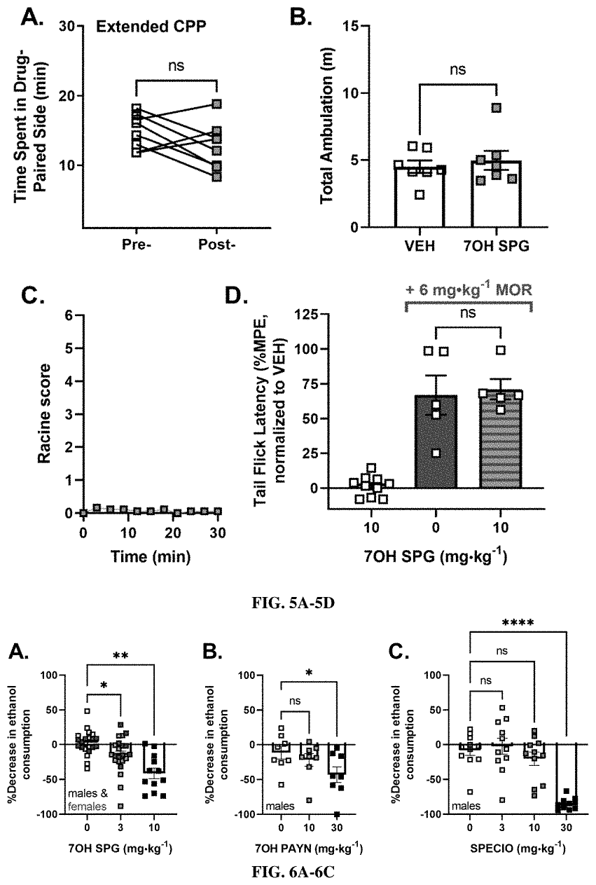

In terms of cAMP inhibition, clear signs of partial agonism were noted for the analogs at the μOP, with paynantheine pseudoindoxyl, 7-hydroxypaynantheine and 7-hydroxyspeciogynine displaying the lowest potency at the μOP (FIG. 3A, FIG. 4A-B, Table 1). 7-hydroxyspeciogynine was the strongest activator at the δOP (FIG. 4A), whereas speciociliatine exhibited the strongest κOP potency out of the tested alkaloids (FIG. 3B, Table 1). Notably, while speciociliatine displayed binding at the δOP, it showed minimal activity at this receptor in regards to cAMP inhibition, suggestive of it acting as antagonist at the δOP (FIG. 3B, FIG. 3E). At the κOP, cAMP inhibition for 7-hydroxypaynantheine was not detected at the tested dose range (FIG. 3F, Table 1).

Furthermore, no β-arrestin2 recruitment was detected for speciociliatine and the pseudoindoxyl and 7-hydroxyl analogs (FIG. 3G-I, Table 1), which is line with the reported G-biased nature of the kratom alkaloids (Gutridge et al., 2020; Kruegel et al., 2016; Váradi et al., 2016).

### C. Kratom Analogs Decrease Ethanol Consumption in a δOP-Dependent Mechanism.

Given the weak μOP potency of 7-hydroxyspeciogynine and 7-hydroxypaynantheine but the clear 10-fold difference in potency at the δOP between the two analogs (FIG. 4A-B), the in vivo potency was assessed for these two alkaloids in modulating volitional alcohol consumption in mice. 7-hydroxypaynantheine was found to be able to significantly reduce alcohol intake at a 30 mg·kg⁻¹ dose (RM 1-way ANOVA, overall effect p=0.0348, F(1.350,9.447)=5.515, with Dunnett's MC to vehicle, p=0.0033) (FIG. 4C) and was slightly less potent than paynantheine, which shows significant reduction at 10 mg·kg⁻¹ (Gutridge et al., 00). At the δOP, 7-hydroxyspeciogynine more potently reduced alcohol intake at a 3 and 10 mg·kg⁻¹ dose in a dose-dependent manner in male and female mice (mixed-effects model, overall effect p=0.0001, F(1.539, 40.80)=13.36, with Dunnett's MC to vehicle, p=0.0165 for the 3 mg·kg⁻¹ dose, p=0.0064 for the 10 mg·kg⁻¹ dose) (FIG. 4D). At doses of 3 and 10 mg·kg⁻¹, 7-hydroxyspeciogynine did not significantly decrease ethanol consumption in δOP KO mice, nor did 30 mg·kg⁻¹ 7-hydroxypaynantheine nor 10 mg·kg⁻¹ paynantheine relative to vehicle (RM 1-way ANOVA, p=0.1901, F(1.966, 15.73)=1.851 (FIG. 4E).

### D. Speciociliatine Modulation of Alcohol Intake is Compounded by Drug-Induced Locomotor Incoordination

Without wishing to be bound by any theory, it is believed that G-protein-biased δOP agonism drives decreased alcohol intake following kratom alkaloid injection. Given this theory, it was not expected that speciociliatine to decrease alcohol intake as it behaves in vitro as a partial agonist for OP and κOP but antagonist at δOP (Table 1). However, speciociliatine significantly decreased ethanol consumption, but only at the 30 mg·kg⁻¹ dose (RM 1-way ANOVA, p<0.0001, F(2.343,23.4)=13.39, with Dunnett's MC, p=0.0005) and with surprising efficacy (an average decrease of 2.5±0.3 g·kg⁻¹ ethanol or a 90±3% reduction, compared to a decrease of 1.2±0.2 g·kg⁻¹ ethanol (40±7%) for 10 mg·kg⁻¹ 7-hydroxyspeciogynine and 1.1±0.3 g·kg⁻¹ ethanol (40±11%) for 30 mg·kg⁻¹ 7-hydroxypaynantheine, FIG. 8) (FIG. 4F). However, the 30 mg·kg¹ dose of speciociliatine also significantly reduced the ability of treated wildtype mice to perform in the rotarod assessment (FIG. 4G). This effect had a rapid onset, where time spent on the device significantly decreased at 5 minutes (one sample t-test, t=3.478, df=7, p=0.0103), with the peak effect occurring between 15 and 30 minutes (t=5.809, df=7, p=0.0007; t=5.344, df=7, p=0.0011, respectively), and the mice fully recovering at 120 minutes (t=1.953, df=7, p=0.0918). The same effect was observed in δOP KO mice (mixed effects model with matching for genotype x timepoint, F(1.941,11.26)=1.930, p=0.1906).

### E. 7-Hydroxyspeciogynine has Lessened Side Effects Due to its Decreased μOP Dependent Pharmacology.

From the cellular and behavioral experiments, 7-hydroxyspeciogynine emerged as the most promising kratom-derived analog for reducing alcohol use, with relatively equal in vivo potency as 7-hydroxymitragynine at the δOP, but lower μOP potency. Next, 7-hydroxyspeciogynine was assessed to exhibit a better side effect profile than 7-hydroxymitragynine due to its limited potency at the μOP. It was found that mice treated with 10 mg·kg⁻¹ 7-hydroxyspeciogynine did not develop conditioned place preference in the ‘extended’ conditioned place preference protocol, which involves four conditioning sessions each for drug and vehicle (paired, two-tailed t-test, t=1.592, df=7, p=0.1554) (FIG. 5A). The same 10 mg·kg⁻¹ dose of 7-hydroxyspeciogynine did not significantly alter ambulation (paired, two-tailed t-test, t=0.7552, df=6, p=0.4787) (FIG. 5B) or induce seizures (FIG. 5C). Akin to 10 mg·kg⁻¹ paynantheine, 10 mg·kg⁻¹ 7-hydroxyspeciogynine did not produce antinociception (paired, two-tailed t-test, t=0.6193, df=9, p=0.5511) or block morphine analgesia (unpaired t-test with Welch's correction, t=0.2660, df=5.994, p=0.7991) (FIG. 5D).

### Example 3: Discussion

Over the past decade, kratom has been reported as a source for naturally occurring, G-protein biased opioidergic alkaloids, and has been investigated for its effects on pain management (Chakraborty & Majumdar, 2021; Matsumoto et al., 2004; Kruegel et al., 2019), opioid withdrawal (Wilson et al., 2020; Wilson et al., 2021), and alcohol abuse (Gutridge et al., 2020), as well as its decreased reward profile relative to traditional opioids (Wilson et al., 2021; Hemby et al., 2019). Here, the effects of kratom alkaloids and synthetic kratom alkaloid derivatives were further probed to obtain a better understanding of its in vivo pharmacology and in search of novel treatment options for alcohol use disorder. Herein, 7-hydroxyspeciogynine was shown to be an effective lead compound with a limited side effect profile.

7-hydroxymitragynine as well as paynantheine were previously demonstrated to decrease alcohol consumption. However, 7-hydroxymitragyinine caused both CPP and hyperlocomotion. It has been well-established that μOP agonism can cause CPP, and that these rewarding effects can be blocked by μOP antagonists (Negus et al., 1993; Piepponen et al., 1997) as well as μOP KO (Matthes et al., 1996). Here, 7-hydroxymitragynine-induced hyperlocomotion was also to be μOP-mediated as it is completely blocked by a dose of naloxone considered to be μOP-selective (Takemori & Portoghese, 1984; Pastor et al., 2005). Since the alcohol-reducing effect of 7-hydroxymitragynine was dependent on δOPs, μOP potency may be a liability when exploring kratom alkaloids as treatment option for AUD. Paynantheine has much lower μOP potency while retaining δOP potency and its ability to decrease alcohol intake in mice at a 10 mg·kg⁻¹ dose without causing hyperlocomotion (Gutridge et al., 2020). In line with the lower μOP potency, it was found that 10 mg·kg⁻¹ paynantheine does not produce place preference in an extended CPP paradigm. In a brief CPP paradigm, however, the same dose of paynantheine induces conditioned place aversion (CPA). Kratom use can lead to seizures (Coonan & Tatum, 2021) and it was noticed that at 30 mg·kg⁻¹, paynantheine induced seizures. δOP agonism can cause seizures (Hong et al., 1998; Broom et al., 2002; Jutkiewicz et al., 2006), however it is reported mostly for δOP agonists that are strong recruiters of β-arrestin, like SNC80 and BW373U86 (Hong et al., 1998; O'Neill et al., 1997; Jutkiewicz et al., 2005). As such, the G-protein-biased paynantheine-induced seizures were surprisingly still present in δOP KO mice. Still, it is possible that mice administered a dose of 10 mg·kg⁻¹ paynantheine did not feel well despite not showing overt signs of seizure activity that could contribute to the observed CPA at this dose. Despite its ability to decrease alcohol consumption with minimal reward liability, 10 mg·kg⁻¹ paynantheine does display a trend towards decreased locomotor activity.

Utilizing the G-protein-biased nature of the kratom alkaloid scaffold, further optimization to discover opioids that have increased δOP potency, with relatively low OP potency. 7-hydroxymitragynine and mitragynine pseudoindoxyl, two previously characterized analogs of mitragynine, had higher δOP as well as μOP affinity and activity in cell lines compared to the indole-based template of mitragynine and showed unique binding poses in computational models (Váradi et al., 2016; Zhou et al., 2021). To extend the structure activity relationship (SAR) to the paynantheine and related speciogynine templates, the hydroxylated and spiropseudoindoxyl variants of these natural products were synthesized. 7-hydroxyspeciogynine and 7-hydroxypaynantheine was identified as having reduced μOP potency but similar δOP potency relative to 7-hydroxymitragynine. In contrast to the mitragynine derived spiropseudoindoxyls, no advantage with respect to potency at the OPs was seen with the pseudoindoxyls derived from payanantheine or speciogynine. Both the novel 7-hydroxyl analogs dose-dependently decreased alcohol consumption, with 7-hydroxyspeciogynine displaying efficacious activity at a dose of 3 mg·kg⁻¹ and 7-hydroxypaynanthiene at a 30 mg·kg⁻¹ dose. The alcohol-modulating effects of these analogs were confirmed as at least partially acting through a δOP-mediated mechanism as statistically significant reductions in alcohol consumption were not observed in δOP KO mice for the two analogs at their effective doses. Additionally, the in vivo potency of these compounds correlates well with their in vitro pharmacology at the δOP where 7-hydroxyspeciogynine is about 0.5-1 log-fold more potent than 7-hydroxypaynantheine (Table 1). While 7-hydroxyspeciogynine displays more potent activity at the μOP relative to 7-hydroxypaynantheine in the GloSensor assay (pIC₅₀s of 6.2±0.3 and 4.7±0.5, respectively), the activity at this receptor is still less potent than 7-hydroxymitragynine (pIC₅₀=7.8±0.1). The G-protein-biased μOP activity of 7-hydroxyspeciogynine likely does not contribute to decreased alcohol use because of the lack of effect in δOP KO mice and because selective activation of μOP G-protein signaling using Oliceridine was shown to not decrease alcohol consumption (Gutridge et al., 2020).

Kratom based natural products, including paynantheine and speciociliatine examined here, have been predicted and shown to have activity at adrenergic 2A, 2B, and 2C receptors and serotonin 2A receptors (Obeng et al., 2020; Ellis et al., 2020; Boyer et al., 2008; Foss et al., 2020). Since we did not screen the kratom analogs for activity at these or other receptors, it is possible that non-δOP activity contributes to the observed alcohol intake modulation. Though there is support for targeting adrenergic and serotonin receptors for treatment of alcohol abuse (Haass-Koffler et al., 2018; Berquist & Fantegrossi, 2021; DiVito & Leger, 2020; Sessa et al., 2021), the data in δOP KO animals supports the hypothesis of the primary role of δOP in decreasing alcohol consumption.

Relative to the GTPyS assay, the GloSensor assay of cAMP inhibition uses recombinant overexpressed cell systems and is amplified relative to measuring G-protein activity directly. As such, without wishing to be bound by any theory, it is believed that the partial agonism for the kratom analogs in vitro were detected and does not resemble how they act in vivo. For example, at the δOP, mitragynine has partial agonism in the cAMP assay but acts as an antagonist in the GTPyS assay (Gutridge et al., 2020; Váradi et al., 2016). Therefore, it may be suggested that the kratom analogs are acting as δOP antagonists in vivo. However, the δOP selective antagonist naltrindole was previously shown to not decrease alcohol intake at 10 mg·kg⁻¹ in this alcohol model (van Rijn et al., 2009), and that δOP KO mice show similar if not increased alcohol intake relative to wild-type mice (van Rijn et al., 2009). Similarly, speciociliatine data counters this argument. At the δOP, speciociliatine binds with a pKi of 5.4±0.1, thus in between the binding affinities of 7-hydroxyspeciogynine and 7-hydroxypayantheine (6.3±0.1 and 4.9±0.2, respectively), yet speciociliatine acts as a δOP antagonist in the cAMP assay. When tested in mice, speciociliatine did cause a significant and sharp decrease in alcohol consumption (90±3% reduction in ethanol consumption, FIG. 8) at a relatively high 30 mg·kg⁻¹ dose, which indicates an off-target effect. In support of this explanation, a 30 mg·kg⁻¹ dose of speciociliatine significantly impairs motor incoordination in wildtype and δOP KO mice, which likely contributes to the effects seen in the alcohol consumption paradigm.

At the μOP, it has recently been demonstrated that a reduction in G-protein efficacy is responsible for lessened adverse side effect profiles, rather than a lack of β-arrestin recruitment (Gillis et al., 2020). This begs the question whether partial agonism rather than full agonism is driving the δOP mediated effects on alcohol intake. The δOP agonist TAN-67 efficaciously reduces alcohol use in the two-bottle choice paradigm, and whilst a full agonist in the cAMP assay, [³⁵S]GTPyS assays have suggested TAN-67 may be a partial agonist (Stanczyk et al., 2019), although an older study found it fully activated [³⁵S]GTPyS (Quock et al., 1997). Thus, the results may provide broader support for the partial agonism hypothesis for beneficial in vivo opioid efficacy.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, such that they provide exemplary procedural or other details supplementary to those set forth herein, are specifically incorporated by reference.
U.S. Pat. No. 4,882,335
Anderson, Practical Process Research & Development—A Guide for Organic Chemists, 2^{nd} ed., Academic Press, New York, 2012.
Bohn et al. Science 286:2495-2498, 1999.
Bu et al., Int J Neurosci 125:56-65, 2015.
Chakraborty et al., J. Med. Chem., 64(18):13873-13892, 2021.
Chen et al., Mol. Pharmacol., 44:8, 1993.
Crooks et al., Bioorganic and Medicinal Chemistry Letters 16:4291-4295, 2006.
Crowley et al., Journal of Medicinal Chemistry 59:11027-11038, 2016.
Dewire et al., J. Pharmacol. Exp. Ther. 344:708-717, 2013.
Faouzi et al., Molecules, 16:25(18):4257, 2020.
Grinnell et al., Synapse 70:395-407, 2016.
Handbook of Pharmaceutical Salts: Properties, and Use, Stahl and Wermuth Eds., Verlag Helvetica Chimica Acta, 2002.
Harding et al., J. Med. Chem 48:4765-4771, 2005.
Hupp & Neumeyer, Tetrahedron Lett. 51:2359-2361, 2010.
Jiang et al., Journal of Medicinal Chemistry 20:1100-1102, 1977.
Kobylecki et al., Journal of Medicinal Chemistry 25:116-120, 1982.
Kranzler, Alcohol & Alcoholism 35:537-547, 2000.
Kruegel et al., Journal of the American Chemical Society 138:6754-6764, 2016.
Li et al., International journal of molecular sciences 10:954-963. 2009.
Lutz and Pfister, J. Receptor Res., 12:267, 1992.
Majumdar et al., Bioorg Med Chem Lett 21:4001-4, 2011.
Manglik et al., Nature 537:185-190, 2016.
Nagase et al., A New Useful Conversion Method of Naltrexone to 14-Deoxynaltrexone, 2006.
Olson et al., Peptides, 10:1253, 1989.
Portoghese et al., Eur. J. Pharmacol., 218:195, 1992.
Raehal et al., J. Pharmacol. Exp. Ther. 314:1195-1201, 2005.
Rankovic et al., Bioorganic & Medicinal Chemistry Letters 26:241-250, 2016.
Reagan-Shaw et al., FASEB J., 22(3):659-661, 2008.
Robinson & Roskamp, Tetrahedron 53:6697-6705, 1997.
Schmid et al., Cell 171:1165-1175.e1113, 2017.
Simon et al., Medical Res. Rev.,
Simon et al., Tetrahedron 50:9757-9768, 1994.
Simon et al., Synthetic Communications 22, 913-921, 1992.
Smith, March's Advanced Organic Chemistry: Reactions, Mechanisms, and Structure, 7^{th} Ed., Wiley, 2013.
Sofuoglu et al., J. Pharmacol. Ther., 257:676, 1991.
Standifer et al., Mol. Pharmacol., 50:293, 1996.
Standifer and Pasternak, Cell Signal, 9:237, 1997.
Tius & Kerr, Journal of the American Chemical Society 114:5959-5966, 1992.
Váradi et al., J Med Chem 59:8381-8397, 2016.
Váradi et al., ACS Chemical Neuroscience 6:1813-1824, 2015.
Yasuda et al., Proc. Natl. Acad. Sci., 90:6736, 1993.
Yuan et al., J Med Chem 56:9156-69, 2013.
Zhang et al., RSC Advances 4:40444-40448, 2014.
Zhang et al., Journal of Medicinal Chemistry 50:2747-2751, 2007.
Zhang et al., J. Med. Chem. 50:2747-2751, 2007.

1. A compound of the formula: 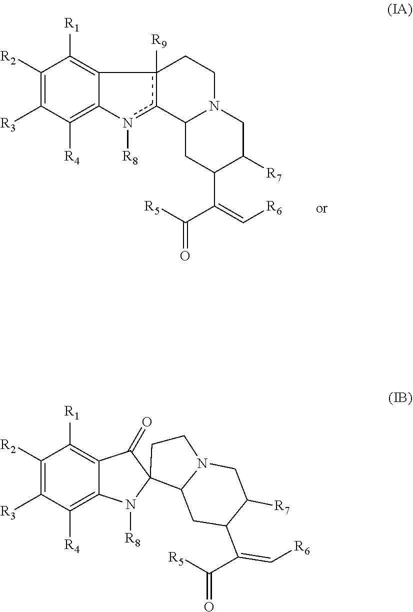
wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₅ is NR′R″ or OR′″ wherein:
R′ and R″ are each independently hydrogen, alkyl_{(C≤8)}, alkenyl_{(C≤8)}, aryl_{(C≤8)}, aralkyl_{(C≤8)}, or a substituted version of any of those groups; a monovalent amine protecting group, or R′ and R″ are taken together and are a divalent amine protecting group;
R′″ is hydrogen, alkyl_{(C≤8)}, alkenyl_{(C≤8)}, aryl_{(C≤8)}, aralkyl_{(C≤8)}, or a substituted version of any of those groups; or a hydroxy protecting group,
R₆ is alkoxy_{(C≤12)} or substituted alkoxy_{(C≤12)};
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
provided that the compound is not a compound of the formula: 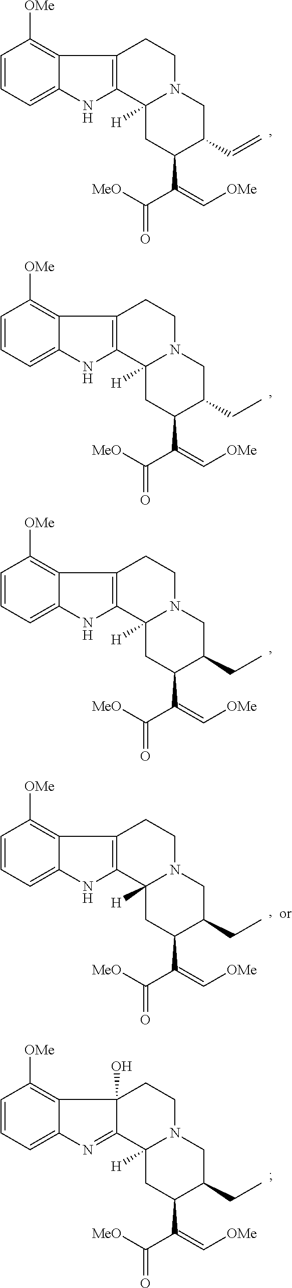 [SMILES: C=C[C@H]1CN2CCc3c([nH]c4cccc(OC)c34)[C@@H]2C[C@@H]1/C(=C\OC)C(=O)OC ; CC[C@H]1CN2CCc3c([nH]c4cccc(OC)c34)[C@@H]2C[C@@H]1/C(=C\OC)C(=O)OC ; CC[C@@H]1CN2CCc3c([nH]c4cccc(OC)c34)[C@@H]2C[C@@H]1/C(=C\OC)C(=O)OC ; CC[C@@H]1CN2CCc3c([nH]c4cccc(OC)c34)[C@H]2C[C@@H]1/C(=C\OC)C(=O)OC ; CC[C@@H]1CN2CC[C@@]3(O)C(=Nc4cccc(OC)c43)[C@@H]2C[C@@H]1/C(=C\OC)C(=O)OC]
or a pharmaceutically acceptable salt thereof.

2. The compound of claim 1 further defined as: 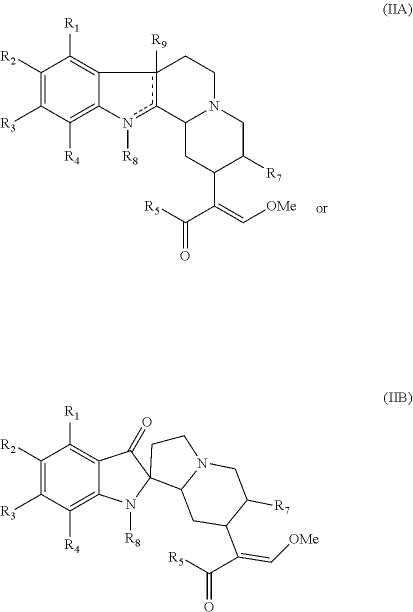
wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₅ is NR′R″ or OR′″ wherein:
R′ and R″ are each independently hydrogen, alkyl_{(C≤8)}, alkenyl_{(C≤8)}, aryl_{(C≤8)}, aralkyl_{(C≤8)}, or a substituted version of any of those groups; a monovalent amine protecting group, or R′ and R″ are taken together and are a divalent amine protecting group;
R′″ is hydrogen, alkyl_{(C≤8)}, alkenyl_{(C≤8)}, aryl_{(C≤8)}, aralkyl_{(C≤8)}, or a substituted version of any of those groups; or a hydroxy protecting group,
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
or a pharmaceutically acceptable salt thereof.

3. The compound of claim 1 further defined as: 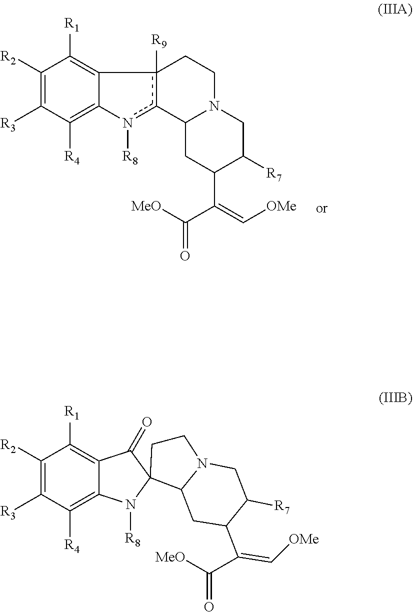
wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
or a pharmaceutically acceptable salt thereof.

4. The compound of claim 1 further defined as: 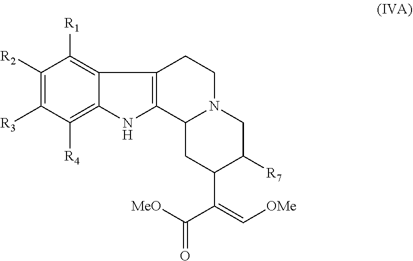
wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
or a pharmaceutically acceptable salt thereof.

5. The compound of claim 1 further defined as: 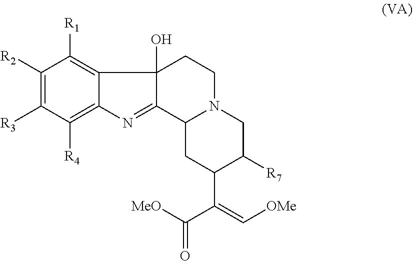
wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
or a pharmaceutically acceptable salt thereof.

6. The compound of claim 1 further defined as: 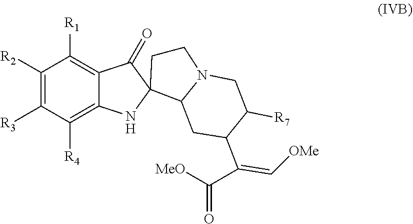
wherein:
R₁, R₂, R₃, or R₄ are each independently selected from hydrogen, halo, hydroxy, alkyl_{(C≤12)}, aryl_{(C≤12)}, heteroaryl_{(C≤12)}, alkoxy_{(C≤12)}, aryloxy_{(C≤12)}, heteroaryl_{(C≤12)}, or a substituted version of any of these groups;
R₇ is alkyl_{(C≤12)}, alkenyl_{(C≤12)}, or alkynyl_{(C≤12)} or a substituted version of these groups;
R₈ is absent, hydrogen, alkyl_{(C≤12)}, or substituted alkyl_{(C≤12)};
R₉ is absent or hydroxy;
or a pharmaceutically acceptable salt thereof.

7. The compound of claim 1, wherein R₆ is alkoxy_{(C≤12)}.

8. The compound of claim 1, wherein R₆ is alkoxy_{(C≤6)}.

9. The compound of claim 1, wherein R₆ is methoxy.

10. The compound of claim 1, wherein R₅ is OR′″.

11. The compound of claim 1, wherein R′″ is alkyl_{(C≤8)} or substituted alkyl_{(C≤8)}.

12. The compound of claim 1, wherein R′″ is alkyl_{(C≤8)}.

13. The compound of claim 1, wherein R′″ is methyl.

14. The compound of claim 1, wherein R′″ is hydrogen.

15. The compound of claim 1, wherein R₅ is NR′R″.

16. The compound of claim 1, wherein R′ is alkyl_{(C≤8)} or substituted alkyl_{(C≤8)}.

17. The compound of claim 1, wherein R′ is alkyl_{(C≤8)}.

18. The compound of claim 1, wherein R′ is methyl.

19. The compound of claim 1, wherein R′ is hydrogen.

20. The compound of claim 1, wherein R″ is alkyl_{(C≤8)} or substituted alkyl_{(C≤8)}.

21. The compound of claim 1, wherein R″ is alkyl_{(C≤8)}.

22. The compound of claim 1, wherein R″ is methyl.

23. The compound of claim 1, wherein R″ is hydrogen.

24. The compound of claim 1, wherein R₉ is absent.

25. The compound of claim 1, wherein R₉ is hydroxy.

26. The compound of claim 1, wherein R₈ is absent.

27. The compound of claim 1, wherein R₈ is hydrogen.

28. The compound of claim 1, wherein R₇ is alkyl_{(C≤12)} or substituted alkyl_{(C≤12)}.

29. The compound of claim 1, wherein R₇ is alkyl_{(C≤12)}.

30. The compound of claim 1, wherein R₇ is alkyl_{(C≤6)}.

31. The compound of claim 1, wherein R₇ is ethyl.

32. The compound of claim 1, wherein R₇ is alkenyl_{(C≤12)} or substituted alkenyl_{(C≤12)}.

33. The compound of claim 1, wherein R₇ is alkenyl_{(C≤12)}.

34. The compound of claim 1, wherein R₇ is alkenyl_{(C≤6)}.

35. The compound of claim 1, wherein R₇ is ethylenyl.

36. The compound of claim 1, wherein R₁ is alkoxy_{(C≤12)} or substituted alkoxy_{(C≤12)}.

37. The compound of claim 1, wherein R₁ is alkoxy_{(C≤12)}.

38. The compound of claim 1, wherein R₁ is alkoxy_{(C≤6)}.

39. The compound of claim 1, wherein R₁ is methoxy.

40. The compound of claim 1, wherein R₁ is hydrogen.

41. The compound of claim 1, wherein R₂ is alkoxy_{(C≤12)} or substituted alkoxy_{(C≤12)}.

42. The compound of claim 1, wherein R₂ is alkoxy_{(C≤12)}.

43. The compound of claim 1, wherein R₂ is alkoxy_{(C≤6)}.

44. The compound of claim 1, wherein R₂ is methoxy.

45. The compound of claim 1, wherein R₂ is hydrogen.

46. The compound of claim 1, wherein R₃ is alkoxy_{(C≤12)} or substituted alkoxy_{(C≤12)}.

47. The compound of claim 1, wherein R₃ is alkoxy_{(C≤12)}.

48. The compound of claim 1, wherein R₃ is alkoxy_{(C≤6)}.

49. The compound of claim 1, wherein R₃ is methoxy.

50. The compound of claim 1, wherein R₃ is hydrogen.

51. The compound of claim 1, wherein R₄ is alkoxy_{(C≤12)} or substituted alkoxy_{(C≤12)}.

52. The compound of claim 1, wherein R₄ is alkoxy_{(C≤12)}.

53. The compound of claim 1, wherein R₄ is alkoxy_{(C≤6)}.

54. The compound of claim 1, wherein R₄ is methoxy.

55. The compound of claim 1, wherein R₄ is hydrogen.

56. The compound of claim 1, wherein the compound is further defined as: 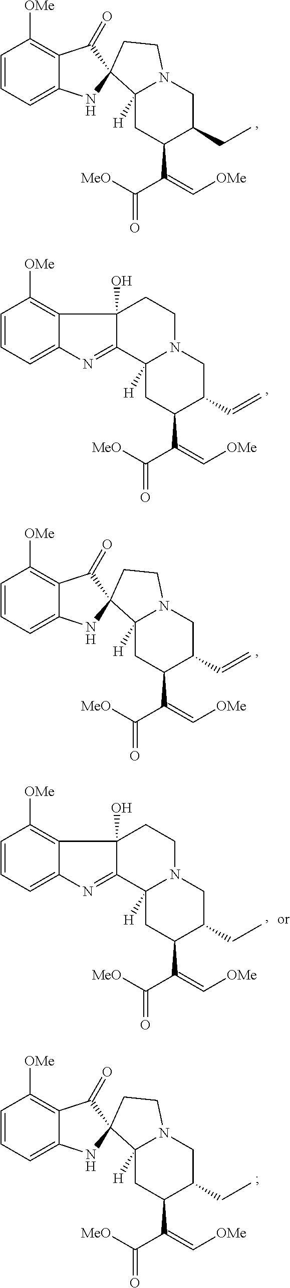 [SMILES: CC[C@@H]1CN2CC[C@]3(Nc4cccc(OC)c4C3=O)[C@@H]2C[C@@H]1/C(=C\OC)C(=O)OC ; C=C[C@H]1CN2CC[C@@]3(O)C(=Nc4cccc(OC)c43)[C@@H]2C[C@@H]1/C(=C\OC)C(=O)OC ; C=C[C@H]1CN2CC[C@]3(Nc4cccc(OC)c4C3=O)[C@@H]2C[C@@H]1/C(=C\OC)C(=O)OC ; CC[C@H]1CN2CC[C@@]3(O)C(=Nc4cccc(OC)c43)[C@@H]2C[C@@H]1/C(=C\OC)C(=O)OC ; CC[C@H]1CN2CC[C@]3(Nc4cccc(OC)c4C3=O)[C@@H]2C[C@@H]1/C(=C\OC)C(=O)OC]
or a pharmaceutically acceptable salt thereof.

57. A pharmaceutical composition comprising:
(A) a compound of claim 1; and
(B) an excipient,

58. A pharmaceutical composition comprising:
(A) a compound of the formula: 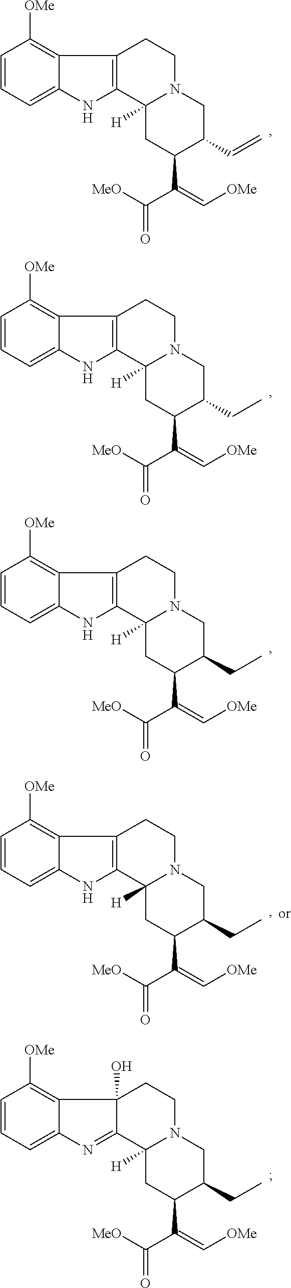 [SMILES: C=C[C@H]1CN2CCc3c([nH]c4cccc(OC)c34)[C@@H]2C[C@@H]1/C(=C\OC)C(=O)OC ; CC[C@H]1CN2CCc3c([nH]c4cccc(OC)c34)[C@@H]2C[C@@H]1/C(=C\OC)C(=O)OC ; CC[C@@H]1CN2CCc3c([nH]c4cccc(OC)c34)[C@@H]2C[C@@H]1/C(=C\OC)C(=O)OC ; CC[C@@H]1CN2CCc3c([nH]c4cccc(OC)c34)[C@H]2C[C@@H]1/C(=C\OC)C(=O)OC ; CC[C@@H]1CN2CC[C@@]3(O)C(=Nc4cccc(OC)c43)[C@@H]2C[C@@H]1/C(=C\OC)C(=O)OC]
(B) an excipient.

59. The pharmaceutical composition of claim 57, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

60. The pharmaceutical composition of claim 57, wherein the pharmaceutical composition is formulated as a unit dose.

61. A method of treating or prevent a disease or disorder comprising administering to a patient in need thereof a compound or composition of claim 1 in a therapeutically effective amount.

62. The method of claim 61, wherein the disease or disorder is alcoholism.

63. The method of claim 61, wherein the patient is a mammal.

64. The method of claim 63, wherein the mammal is a human.

65. The method of claim 61, wherein the disease or disorder is associated with the δ opioid receptor.

66. The method of claim 61, wherein the compound or composition results in greater modulation of δ opioid receptor compared to μ opioid receptor.

67. A method of reducing alcohol composition in a patient comprising administering to the patient a therapeutically effective amount of a compound or composition of claim 1.

68. The method of claim 67, wherein the patient is a mammal.

69. The method of claim 68, wherein the mammal is a human.

70. The method of claim 67, wherein the compound or composition is associated with the δ opioid receptor.

71. The method of claim 67, wherein the compound or composition results in greater modulation of δ opioid receptor compared to μ opioid receptor.

72. A method of modulating the activity of a δ opioid receptor comprising contacting the δ opioid receptor with a compound or composition of claim 1.

73. The method of claim 72, wherein the method is performed in vitro.

74. The method of claim 72, wherein the method is performed in vivo.

75. The method of claim 72, wherein the method is performed ex vivo.

76. The method of claim 72, wherein the compound or composition results in greater modulation of δ opioid receptor compared to μ opioid receptor.
